(19) United States
(12) Patent Application Publication
(10) Pub. No.: US 2026/0060736 A1
(43) Pub. Date: Mar. 5, 2026

(54) PFA CATHETER ELECTRODE CONNECTIONS

(71) Applicant: Boston Scientific Scimed, Inc., Maple Grove, MN (US)

(72) Inventors: James Holmberg, Champlin, MN (US); Cristian Clarke, Fridly, MN (US); Nathan Paul Hagstrom, Blaine, MN (US)

(21) Appl. No.: 19/310,557

(22) Filed: Aug. 26, 2025

Related U.S. Application Data
(60) Provisional application No. 63/688,716, filed on Aug. 29, 2024.

(51) Int. Cl. A61B 18/00

(52) U.S. Cl. CPC A61B 18/00; A61B 2018/0016; A61B 2018/00178; A61B 2018/00202; A61B 2018/00351; A61B 2018/00577; A61B 2018/00613; A61B 2018/00839

(57) ABSTRACT

A catheter for ablating cardiac tissue through irreversible electroporation is disclosed. The catheter includes an elongated shaft having a distal region, in which the elongated shaft defining a longitudinal axis, and a set of splines extending from the distal region of the shaft. The set of splines are configured for translation along the longitudinal axis to transition between a collapsed configuration and an expanded configuration. Each spline forms a loop in the expanded configuration. Each spline of the set of splines includes an electrode assembly having a flexible circuit within the spline. The flexible circuit includes lead traces electrically coupled to electrodes on a surface of the spline.

### CROSS REFERENCE TO RELATED APPLICATIONS

This application claims priority to U.S. Provisional Patent Application No. 63/688,716 entitled “PFA CATHETER ELECTRODE CONNECTIONS,” filed Aug. 29, 2024, which is hereby incorporated by reference in its entirety.

### TECHNICAL FIELD

The present disclosure relates to medical systems and methods for ablating tissue in a patient. More specifically, the present disclosure relates to medical systems and methods for ablation of tissue by electroporation.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electrical field is applied to cells to increase the permeability of the cell membrane. The electroporation can be reversible or irreversible, depending on the strength of the electric field. If the electroporation is reversible, the increased permeability of the cell membrane can be used to introduce chemicals, drugs, and/or deoxyribonucleic acid (DNA) into the cell, prior to the cell healing and recovering. If the electroporation is irreversible, the affected cells are killed through apoptosis.

Irreversible electroporation can be used as a nonthermal ablation technique. In irreversible electroporation, trains of short, high voltage pulses are used to generate electric fields that are strong enough to kill cells through apoptosis. In ablation of cardiac tissue, irreversible electroporation can be a safe and effective alternative to the indiscriminate killing of thermal ablation techniques, such as RF ablation and cryoablation. Irreversible electroporation can be used to kill targeted tissue, such as myocardium tissue, by using an electric field strength and duration that kills the targeted tissue but does not permanently damage other cells or tissue, such as non-targeted myocardium tissue, red blood cells, vascular smooth muscle tissue, endothelium tissue, and nerve cells.

Electrophysiological procedures, which include catheter ablation to treat a variety of heart conditions such as supraventricular and ventricular arrhythmia, involve a visualization of the heart and heart activity. Electroanatomical mapping is a visualization technique that allows a clinician to accurately determine the location of an arrhythmia, define cardiac geometry in three dimensions, delineate areas of anatomic interest, and permits imaging of the catheter assembly for positioning and manipulation. For instance, electroanatomical mapping involves the mapping of electrical activity in the heart based on cardiac signals, such as at various locations on the endocardium surface, to identify the site of origin of the arrhythmia followed by a targeted ablation of the site.

To perform such cardiac mapping, a catheter with an electrode assembly at a distal tip of the catheter can be inserted into the patient's heart chamber. In some examples of mapping, physiological signals from electrical activity of the heart are acquired with electrodes after the tip is in stable and steady contact with the endocardium surface of a particular heart chamber. Alternatively, or additionally, physiological signals can be detected by non-contact electrodes along with information on chamber anatomy and relative electrode location to provide physiological information regarding the endocardium of the heart chamber. The locations of the physiological signals are determined, such as via location sensors on or near the electrode assembly. Location and electrical activity are measured, such as sequentially on a point-by-point basis in some examples, at about fifty to several hundred points on the internal surface of the heart to construct an electroanatomical map of the heart. The generated electroanatomical map can serve several purposes, such as the basis to decide on a therapeutic course of action like tissue ablation, which can be applied to alter the propagation of electrical activity in the heart and to restore normal heart rhythm.

### SUMMARY

In Example 1, a catheter for ablating cardiac tissue through irreversible electroporation, the catheter comprising: an elongated shaft having a distal region, the elongated shaft defining a longitudinal axis; and a set of splines extending from the distal region of the shaft, the set of splines configured for translation along the longitudinal axis to transition between a collapsed configuration and an expanded configuration, wherein each spline forms a loop in the expanded configuration; wherein each spline of the set of splines includes an electrode assembly comprising a flexible circuit within the spline, the flexible circuit including a plurality of lead traces electrically coupled to a plurality of electrodes on a surface of the spline.

In Example 2, the catheter of Example 1, wherein the electrode assembly includes a plurality of electrode assemblies formed on each spline, the plurality of electrode assemblies including an ablation electrode assembly and a sensing electrode assembly, wherein the flexible circuit includes a set of ablation lead traces of the plurality of lead traces electrically coupled to a plurality of ablation electrodes of the plurality of electrodes and wherein the flexible circuit includes a set of sensing lead traces of the plurality of lead traces electrically coupled to a plurality of sensing electrodes of the plurality of electrodes.

In Example 3, the catheter of Example 2, wherein a distal-most sensing electrode is distal to a distal-most ablation electrode and a proximal-most sensing electrode is on the spline.

In Example 4, the catheter of any of Examples 2-3, wherein each spline includes an equal number of sensing electrodes and ablation electrodes.

In Example 5, the catheter of any of Examples 1-4, wherein each of the plurality of electrodes is individually addressable.

In Example 6, the catheter of Example 1, wherein the electrodes of the plurality of electrodes are configurable as at least one of an ablation electrode and a sensing electrode.

In Example 7, the catheter of any of Examples 1-6 wherein the spline defines a major spline lumen, and the flexible circuit is disposed within the major spline lumen.

In Example 8, the catheter of any of Examples 1-7, wherein that spline includes a shape memory strut.

In Example 9, the catheter of Example 8, wherein the shape memory strut is shape set in the expanded configuration.

In Example 10, the catheter of any of Examples 1-9, wherein the set of splines in the expanded configuration are arranged as a set of electrically isolated loops.

In Example 11, the catheter of any of Examples 1-10, wherein the set of splines are arranged to helically rotate about the longitudinal axis.

In Example 12, the catheter of any of Examples 1-11, and further comprising a distal cap coupled to a distal portion of each spline of the set of splines.

In Example 13, the catheter of Example 12, wherein the loop each spline forms includes a first concave curve facing the distal cap, a second concave curve facing the longitudinal axis, and a third concave curve facing the distal end of the shaft.

In Example 14, the catheter of any of Examples 1-13, wherein the electrodes are outwardly-facing on the surface of the spline.

In Example 15, the catheter of any of Examples 1-14, and further comprising a electroporation console electrically coupled to the catheter to deliver a source of ablation energy.

In Example 16, a catheter for ablating cardiac tissue through irreversible electroporation, the catheter comprising: an elongated shaft having a distal region, the elongated shaft defining a longitudinal axis; and a set of splines extending from the distal region of the shaft, the set of splines configured for translation along the longitudinal axis to transition between a collapsed configuration and an expanded configuration, wherein each spline forms a loop in the expanded configuration; wherein each spline of the set of splines includes an electrode assembly comprising a flexible circuit within the spline, the flexible circuit including a plurality of lead traces electrically coupled to a plurality of electrodes on a surface of the spline.

In Example 17, the catheter of Example 16, wherein the electrode assembly includes a plurality of electrode assemblies formed on each spline, the plurality of electrode assemblies including an ablation electrode assembly and a sensing electrode assembly, wherein the flexible circuit includes a set of ablation lead traces of the plurality of lead traces electrically coupled to a plurality of ablation electrodes of the plurality of electrodes and wherein the flexible circuit includes a set of sensing lead traces of the plurality of lead traces electrically coupled to a plurality of sensing electrodes of the plurality of electrodes.

In Example 18, the catheter of Example 17, wherein a distal-most sensing electrode is distal to a distal-most ablation electrode and a proximal-most sensing electrode is on the spline.

In Example 19, the catheter of Example 17, wherein each spline includes an equal number of sensing electrodes and ablation electrodes.

In Example 20, the catheter of Example 16, wherein each of the plurality of electrodes is individually addressable.

In Example 21, the catheter of Example 16, wherein the electrodes of the plurality of electrodes are configurable as at least one of an ablation electrode and a sensing electrode.

In Example 22, the catheter of Example 16 wherein the spline defines a major spline lumen, and the flexible circuit is disposed within the major spline lumen.

In Example 23, the catheter of Example 16, wherein that spline includes a shape memory strut.

In Example 24, the catheter of Example 23, wherein the shape memory strut is shape set in the expanded configuration.

In Example 25, the catheter of Example 16, wherein the set of splines in the expanded configuration are arranged as a set of electrically isolated loops.

In Example 26, the catheter of Example 16, wherein the set of splines are arranged to helically rotate about the longitudinal axis.

In Example 27, the catheter of Example 16, and further comprising a distal cap coupled to a distal portion of each spline of the set of splines.

In Example 28, the catheter of Example 27, wherein the loop each spline forms includes a first concave curve facing the distal cap, a second concave curve facing the longitudinal axis, and a third concave curve facing the distal end of the shaft.

In Example 29, the catheter of Example 16, wherein the electrodes are outwardly-facing on the surface of the spline.

In Example 30, a catheter for ablating cardiac tissue through irreversible electroporation, the catheter comprising: an elongated shaft having a distal region, the elongated shaft defining a longitudinal axis; a set of splines extending from the distal region of the shaft; and a set of splines extending from the distal region of the shaft; a distal cap coupled to a distal portion of each spline of the set of splines, the set of splines configured for translation along the longitudinal axis to transition between a collapsed configuration and an expanded configuration, wherein in the expanded configuration, each spline forms a loop having a first concave curve facing the distal cap, a second concave curve facing the longitudinal axis, and a third concave curve facing the distal end of the shaft; wherein each spline of the set of splines includes an electrode assembly formed on that spline, the electrode assembly comprising a flexible circuit within that spline, the flexible circuit including a plurality of lead traces electrically coupled to a plurality of outwardly-facing electrodes on a surface of the spline.

In Example 31, the catheter of Example 30, wherein the electrode assembly includes a plurality of electrode assemblies formed on each spline, the plurality of electrode assemblies including an ablation electrode assembly and a sensing electrode assembly, wherein the flexible circuit includes a set of ablation lead traces of the plurality of lead traces electrically coupled to a plurality of ablation electrodes of the plurality of electrodes and wherein the flexible circuit includes a set of sensing lead traces of the plurality of lead traces electrically coupled to a plurality of sensing electrodes of the plurality of electrodes.

In Example 32, the catheter of Example 31, wherein a distal-most sensing electrode is distal to a distal-most ablation electrode and a proximal-most sensing electrode is on the spline and wherein each spline includes an equal number of sensing electrodes and ablation electrodes.

In Example 33, a catheter for ablating cardiac tissue through irreversible electroporation, the catheter comprising: an elongated shaft having a distal region, the elongated shaft defining a longitudinal axis; a set of splines extending from the distal region of the shaft; and a distal cap coupled to a distal portion of each spline of the set of splines, the set of splines configured for translation along the longitudinal axis to transition between a collapsed configuration and an expanded configuration, wherein in the expanded configuration, each spline forms a loop having a first concave curve facing the distal cap, a second concave curve facing the longitudinal axis, and a third concave curve facing the distal end of the shaft; wherein each spline of the set of splines includes an electrode assembly formed on the spline, the electrode assembly including an ablation electrode assembly and a sensing electrode assembly, the electrode assembly comprising a flexible circuit within the spline, the flexible circuit including a plurality of lead traces electrically coupled to a plurality of electrodes on a surface of the spline, wherein the flexible circuit includes a set of ablation lead traces of the plurality of lead traces electrically coupled to a plurality of ablation electrodes of the plurality of electrodes and wherein the flexible circuit includes a set of sensing lead traces of the plurality of lead traces electrically coupled to a plurality of sensing electrodes of the plurality of electrodes.

In Example 34, the catheter of Example 33, wherein the set of splines in the expanded configuration are arranged as a set of electrically isolated loops.

In Example 35, the catheter of Example 33, wherein the set of splines are arranged to helically rotate about the longitudinal axis.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system.

FIG. 2A is a side view of a catheter suitable for use in the electrophysiological system of FIG. 1, in which the catheter is in a collapsed configuration.

FIG. 2B is a side view of a distal end of the catheter of FIG. 2A in the collapsed configuration.

FIG. 2C is a perspective view of the distal end of the catheter of FIG. 2A in an intermediate configuration.

FIG. 2D is a perspective view of the distal end of the catheter of FIG. 2A in an expanded configuration.

FIG. 2E is a side view of the distal end of the catheter of FIG. 2A in the expanded configuration.

FIG. 2F is a front view of the distal end of the catheter of FIG. 2B in the expanded configuration.

FIG. 2G is a cross-sectional view of a spline on the distal end of the catheter of FIG. 2A.

FIG. 3A is a cutaway side view of an example of a spline configured for use in the catheter of FIG. 2A.

FIG. 3B is a cross-sectional view of the spline of FIG. 3A.

FIG. 4 is a cross-sectional view of another example spline configured for use in the catheter of FIG. 2A.

FIG. 5 is a cross-sectional view of another example spline configured for use in the catheter of FIG. 2A.

FIG. 6 is a cross-sectional view of another example spline configured for use in the catheter of FIG. 2A.

FIG. 7 is a cross-sectional view of another example spline configured for use in the catheter of FIG. 2A.

FIG. 8 is a perspective view of an example electrode configured for use in the catheter of FIG. 2A.

FIG. 9 is cutaway perspective view of an example spline of the distal end of the catheter of FIG. 2A.

FIG. 10 is a cutaway perspective view of another example spline of the distal end of the catheter of FIG. 2A.

FIG. 11 is a cutaway side view of another example spline configured for use in the catheter of FIG. 2A.

FIG. 12A is side view of a distal end of the catheter of FIG. 2A in the collapsed configuration of an example electrode array having a navigational sensor.

FIG. 12B is a cutaway perspective view of an example spline of the example electrode array of FIG. 12A.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

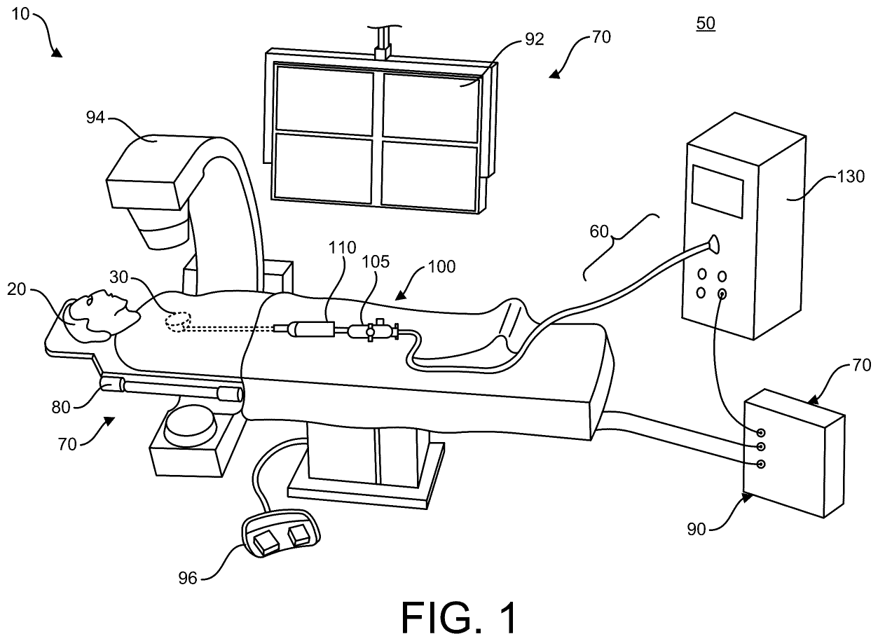

FIG. 1 illustrates an example clinical setting 10 for treating a patient 20, such as for treating a heart 30 of the patient 20, using an electrophysiology system 50, in accordance with the disclosure. The electrophysiology system 50 includes an electroporation catheter system 60 and an electro-anatomical mapping (EAM) system 70. The example electroporation catheter system 60 includes an electroporation catheter 105, an introducer sheath 110, and an electroporation console 130. Additionally, the electroporation catheter system 60 includes various connecting elements, such as cables, that operably connect the components of the electroporation catheter system 60 to one another and to the components of the EAM system 70. In general, the EAM mapping system 70 includes a localization field generator 80, a mapping and navigation controller 90, and a display 92. Also, the clinical setting 10 can include additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1. Other arrangements of connecting elements, including wireless connecting elements, are contemplated.

The electroporation catheter system 60 is configured to deliver electric field energy to targeted tissue in the patient's heart 30 to create cell death in tissue, for example, rendering the tissue incapable of conducting electrical signals. Also, the electroporation catheter system 60 is configured to generate, based on models of electric fields, graphical representations of the electric fields that can be produced using the electroporation catheter 105 and to overlay, on the display 92, the graphical representations of the electric fields or expected or predicted lesions on an anatomical map of the patient's heart to aid a user in planning ablation by irreversible electroporation using the electroporation catheter 105 prior to delivering energy. In embodiments, the electroporation catheter system 60 is configured to generate the graphical representations of the electric fields based on characteristics of the electroporation catheter 105 and the position of the electroporation catheter 105 in the patient 20, such as in the heart 30 of the patient 20. The electroporation catheter system 60 is configured to generate the graphical representations of the electric fields based on characteristics of the electroporation catheter 105 and the position of the electroporation catheter 105 in the patient 20, such as in the heart 30 of the patient 20, and the characteristics of the tissue surrounding the catheter 105, such as measured impedances of the tissue.

The introducer sheath 110 is operable to provide a delivery conduit through which the electroporation catheter 105 can be deployed to the specific target sites within the patient's heart 30. Access to the patient's heart can be obtained through a vessel, such as a peripheral artery or vein. Once access to the vessel is obtained, the electroporation catheter 105 can be navigated to within the patient's heart, such as within a chamber of the heart.

The example electroporation catheter 105 includes an elongated catheter shaft and distal end configured to be deployed proximate target tissue, such as within a chamber of the patient's heart. The distal end includes an electrode array to effect treatment. The catheter 105 is capable of being formed into a plurality of configurations. For example, if the distal end region of the catheter is within the patient's vasculature or is within a sheath as a catheter assembly, such as to travel to the patient to the chamber of the heart, the electrode array is in a collapsed configuration to fit within the sheath. Once the catheter has reached the destination in the chamber of the heart, for example, or the sheath is retracted from the distal region of the catheter 105 (or the shaft catheter is extended past the sheath), and the electrode array is arranged in an expanded configuration for use. In one embodiment, the electrode array can assume other configurations, such as an intermediate configuration between the collapsed and expanded configurations, such as an additional use configuration.

The electrode array includes an electrode assembly comprising a plurality of electrodes. For example, the electrode assembly includes a plurality of spaced-apart electrodes or multiple spaced-apart sets or groups of spaced-apart electrodes. In some examples, an electrode, such as a plurality of spaced-apart electrodes, can be deployed on the catheter shaft in addition to electrodes on the electrode array. In one example, the plurality of electrodes can be formed of a conductive, solid-surface, biocompatible material and are spaced-apart across electrical insulators. Each of the plurality of electrodes is electrically coupled to an associated elongated lead conductor that extend along the shaft to a catheter proximal end. In one example, each electrode of the spaced-apart electrodes corresponds with a separate, single lead conductor. In another example, a plurality of electrodes may be coupled to a single lead conductor. Other configurations are contemplated. The plurality of lead conductors can be electrically insulated from one another within an electrically insulating sheath along the catheter shaft, such as with an electrically insulating polymer sheath. The lead conductors can be electrically coupled to plug in the proximal region of the electroporation catheter 105, such as a plug configured to be mechanically and electrically coupled to the electroporation console 130 and the EAM system 70, for example, either directly or via intermediary electrical conductors such as cabling.

The electrode assemblies and associated electrodes are configured for, among other things, sensing cardiac electrical signals, ablation, localization of the electrode assembly within the patient anatomy such as via the EAM system 70, signal reference, and to determine proximity to target tissue within the anatomy. In some embodiments, the catheter 105 is configured for cardiac mapping, and the electrodes are sensing, or mapping, electrodes configured to be used to collect physiological (electrical) signals to be used to generate electroanatomical maps. An example of a physiological signal that the sensing electrode can acquire includes an intracardiac electrogram (ECG) signal. In some embodiments, the catheter 105 can be a mapping and ablation catheter, and the electrodes can include ablation electrodes, or an ablation electrode assembly, that are configured to deliver ablation electric field energy and sensing electrodes, or a sensing electrode assembly, for mapping purposes. The ablation electrodes in embodiments of an electroporation catheter are configured to receive pulsed electrical signals or waveforms from the console 130 and create pulsed electric fields sufficient to ablate target tissue via irreversible electroporation. The sensing electrodes in the electrode assembly can be electrically coupled to a one or more lead conductors that extends the length of the shaft that are configured to carry an electrical signal received at the sensing electrode. In some examples, an electrode in the electrode assembly can be configured to only perform an ablation or the electrode in the electrode assembly can be configured to only perform mapping. In some examples, an electrode can operate as an ablation electrode in an ablation mode of the electrophysiology system 50 and as a sensing electrode in a sensing or mapping mode of the system 50. Some examples of mapping and ablation catheters are smaller in profile or in the volume of the electrode assembly than catheters that just perform mapping, and clinicians can map a given location within the heart with fewer passes across the chamber with mapping catheters than with mapping and ablation catheters.

In one example, the electroporation console 130 is configured to provide an electrical signal, such as a plurality of concurrent or space-apart-time electrical signals, to the electrically connected electroporation catheter 105 along lead conductors to the spaced-apart electrodes. The spaced-apart electrodes are configured to generate a selected electrical field proximate the target tissue, based on the electrical signals from the electroporation console 130, to effect electroporation.

A selected electrical field can be generated with the electrodes configured as ablation electrodes to effect electroporation. A first ablation electrode, or first group of ablation electrodes, can be selected to be an anode and a different, second ablation electrode, or second group of ablation electrodes, can be selected to be a cathode, such that electrical fields can be generated between the anode and cathode based on signals, such as pulses, provided to the ablation electrodes from the electroporation console 130. The console 130 provides electric pulses of different lengths and magnitudes to the ablation electrodes on the catheter 105. The electric pulses can be provided in a continuous stream of pulses or in multiple, separate trains of pulses. Pulse parameters of interest include the number of pulses, the duty cycle of the pulses, the spacing of pulse trains, the voltage or magnitude of the pulses including the peak voltages, and the duration of the voltages. For example, the console 130 can select two or more ablation electrodes of the electrode assembly and provides pulses to the selected electrodes to generate electric fields between the selected electrodes to provide pulsed field ablation (PFA). For example, PFA can be performed with monophasic waveforms and biphasic waveforms. Without being bound to a particular theory, electric field strengths in the range of generally 200-250 volts per centimeter (V/cm) with microsecond-scale pulse duration have been demonstrated to provide reversible electroporation in cardiac tissue. Electric field strengths at approximately 400 V/cm have been demonstrated to provide irreversible electroporation in cardiac tissue of interest, such as targeted myocardium tissue and endocardium tissue, with demonstrable sparing of red blood cells, vascular smooth muscle tissue, endothelium tissue, nerves and other non-targeted proximate tissue.

Additionally, the electrode assembly on catheter 105 can be operated in a selected mode such as monopolar mode or bipolar mode. During monopolar operation of the catheter 105, an ablation electrode, a group of ablation electrodes, or the entire electrode assembly are configured as one of an anode or a cathode. None of the electrodes in the electrode assembly are configured as a the other of the cathode or the anode. Instead, the other of the cathode or the anode is provided in the form of a pad dispersive electrode located on the patient, typically on the back, buttocks, or other suitable anatomical location during electroporation. An electrical field is formed between an activated electrode of the electrode assembly and the pad dispersive electrode. During bipolar operation of the catheter 105, a first set of one or more electrodes of the electrode assembly, is configured as the anode and a second set of one or more electrodes of the electrode assembly, is configured as the cathode, to generate the electric field. In this example, a pad dispersive electrode is not used, and the electrical field is not extended in the patient's body, but rather through a localized portion of tissue proximate the electrode assembly. For example, the electrodes on the ablation electrode assembly are configured as the one of the anode or cathode and electrodes on the shaft proximate the distal end are configured as the other of the cathode or anode.

The EAM system 70 is operable to track the location of the various components of the electroporation catheter system 60, and to generate high-fidelity three-dimensional anatomical and electro-anatomical maps of the heart, including portions of the heart such as cardiac chambers of interest or other structures of interest such as the sinoatrial node or atrioventricular node. In one illustrative example, the EAM system 70 can include the OPAL™ HDx mapping system marketed by Boston Scientific Corporation. Also, the mapping and navigation controller 90 of the EAM system 70 includes one or more controllers, such as microprocessors or computers, that execute code out of memory to control or perform functional aspects of the EAM system 70, in which the memory, can be part of the one or more controllers, microprocessors, computers, or part of a memory device accessible through a computer network.

The EAM system 70 generates a localization field, via the field generator 80, to define a localization volume about the heart 30, and a location sensor or sensing element on a tracked device, such as sensors on the electroporation catheter 105, generate an output that can be processed by the mapping and navigation controller 90 to track the location of the sensor, and consequently, the corresponding device, within the localization volume. In the illustrated example, the device tracking is accomplished using magnetic tracking techniques, in which the field generator 80 is a magnetic field generator that generates a magnetic field defining the localization volume, and location sensors on the tracked devices are magnetic field sensors.

In other examples, impedance tracking methodologies may be employed to track the locations of the various devices. In such examples, the localization field is an electric field generated, for example, by an external field generator arrangement, such as surface electrodes, by intra-body or intra-cardiac devices, such as an intracardiac catheter, or both. In these examples, the location sensing elements can constitute electrodes on the tracked devices that generate outputs received and processed by the mapping and navigation controller 90 to track the location of the various location sensing electrodes within the localization volume.

The EAM system 70 can be equipped for both magnetic and impedance tracking capabilities. In such examples, impedance tracking accuracy can, in some instances be enhanced by first creating a map of the electric field induced by the electric field generator within the cardiac chamber of interest using a probe equipped with a magnetic location sensor, as is possible using the OPAL HDx™ mapping system.

Regardless of the tracking methodology employed, the EAM system 70 utilizes the location information for the various tracked devices, along with cardiac electrical activity acquired by, for example, the electroporation catheter 105 or another catheter or probe equipped with sensing electrodes, to generate, and display via the display 92, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map or the electro-anatomical map.

Each cardiac physiological (electrical) signal can include several intracardiac electrograms (EGMs) sensed within a patient's heart and may include any number of features that may be ascertained by aspects of the system 50. Examples of cardiac physiological signal features include activation times, activations, activation waveforms, filtered activation waveforms, minimum voltage values, maximum voltages values, maximum negative time-derivatives of voltages, instantaneous potentials, voltage amplitudes, dominant frequencies, and peak-to-peak voltages. A cardiac physiological signal feature can refer to one or more features extracted from one or more cardiac physiological signals, derived from one or more features that are extracted from one or more cardiac physiological signals. Additionally, a representation, on a cardiac or a surface map, of a cardiac physiological signal feature may represent one or more cardiac physiological signal features, an interpolation of several cardiac physiological signal features. Each cardiac physiological signal also can be associated with a set of respective position coordinates that corresponds to the location at which the cardiac physiological signal was sensed. Each of the respective position coordinates for the sensed cardiac physiological signals can include three-dimensional Cartesian coordinates, polar coordinates, or another coordinate system. The cardiac physiological signals may be sensed on the cardiac surfaces, and the respective position coordinates can be on the endocardial surface, epicardial surface, in the mid-myocardium of the patient's heart, or in a vicinity.

During a signal-acquisition stage of a cardiac mapping procedure, the catheter 105 is displaced to multiple locations within the heart chamber into which the catheter 105 is inserted. At each location to which the catheter 105 is moved, the electrodes and sensors acquire physiological signals resulting from the electrical activity in the heart along with positional, or spatial, information of the catheter 105. The spatial information is used in building a three-dimensional grid of the anatomy during mapping. To perform a mapping procedure and reconstruct physiological information on the endocardium surface, the EAM system 70 may align a coordinate system of the catheter 105 with the endocardium surface's coordinate system, or vice versa. Alternatively, or additionally, the grid may be used to capture EGMs, and select mapping values based on statistical distributions associated with nodes of the grid. The EAM system 70 also can perform post-processing operations on the physiological information to extract and display useful features of the information to the operator of the system 50.

In generating an example electroanatomical map, a data stream including multiple signals, such as signals received from the mapping electrodes of the catheter 105, is input into the EAM system 70. During the automated electroanatomical mapping process, the data stream provides a collection of physiological and location signals that serve as an input to the mapping process. The signals may be collected directly by the mapping system, obtained from another system using an analog or digital interface, or both. The data stream can include signals such as unipolar and/or bipolar intracardiac EGMs, surface electrocardiograms (ECGs), electrode location information originating from one or more of a variety of methodologies, tissue proximity information, catheter force information, catheter to tissue contact information, catheter temperature, acoustic information, catheter electrical coupling information, catheter deployment shape information, electrode properties, respiration phase, blood pressure, and other physiological information. For the generation of specific types of maps, one or more signals may be used as one or more references to trigger and align the data stream relative to a cycle or clock, which can be used to create beat datasets. Beat metrics can be determined from the beat datasets. A beat acceptance process can be applied to determine which beat datasets will make up a map dataset. The map dataset may be stored in association with a three-dimensional grid that is dynamically generated during data acquisition.

Surface geometry data of the cardiac surface is generated, such as generated concurrently, during the data acquisition process using acceptance metrics employing a surface geometry construction process. This process constructs surface geometry using data such as electrode locations and catheter shape contained in the data stream. Additionally, or alternatively, previously collected surface geometry of the cardiac surface can be used as an input to surface geometry data. Previously collected geometry may have been collected using a different map dataset or using a different modality such as computerized tomography (CT), magnetic resonance imaging (MRI), ultrasound, or rotational angiography and registered to the catheter locating system. A surface map generation process is employed to generate surface map data from the map dataset and surface geometry data.

The depiction of the electrophysiology system 50 shown in FIG. 1 is intended for illustration or a general overview of the various components of the system 50 and is not intended to imply that the disclosure is limited to any set of components or arrangement of the components. For example, additional hardware components, such as breakout boxes or workstations, can be included in the electrophysiology system 50.

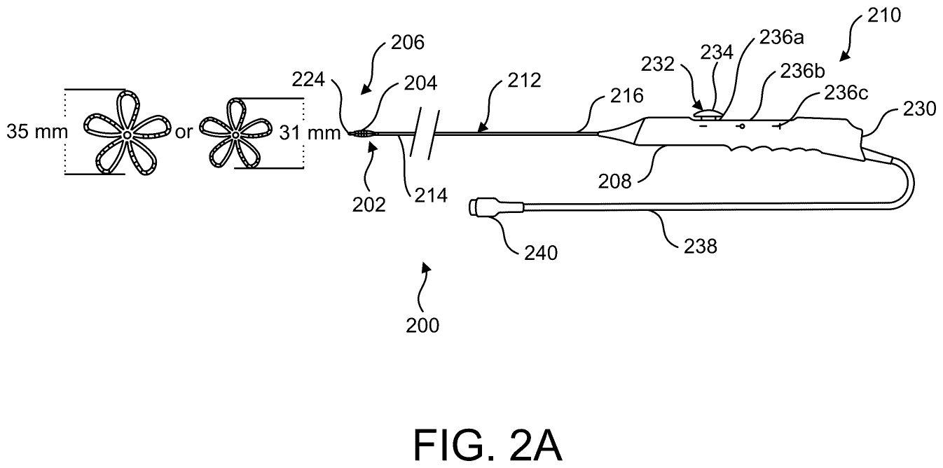

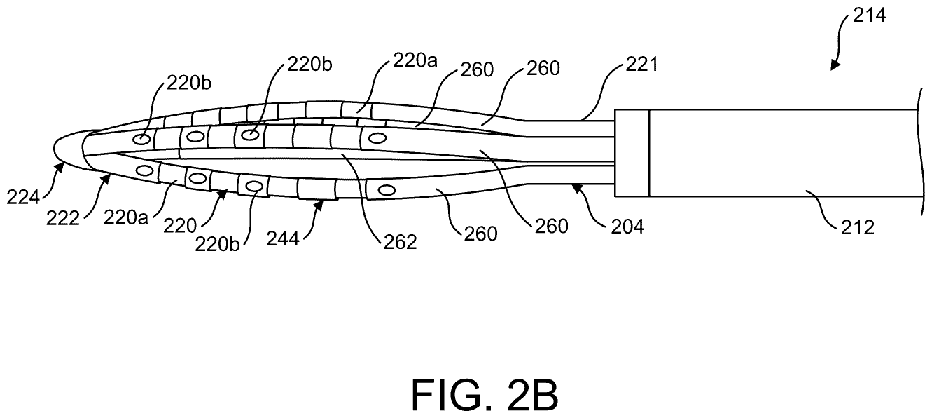

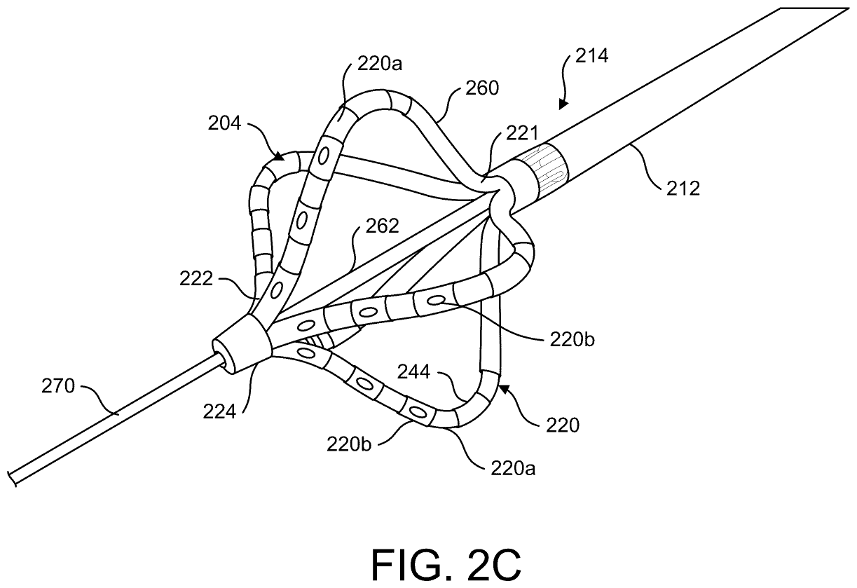

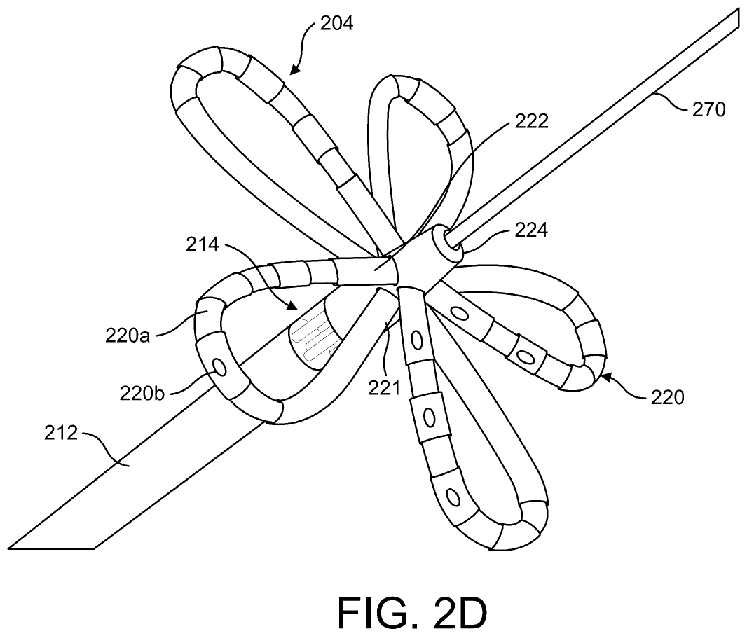

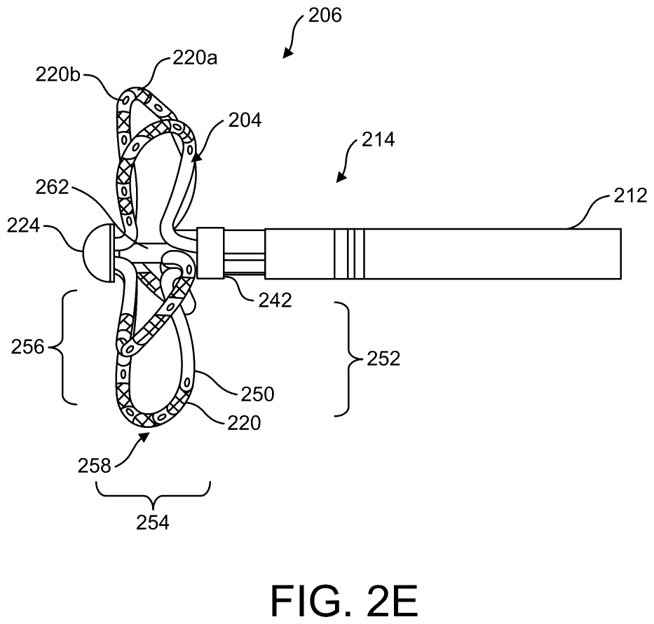

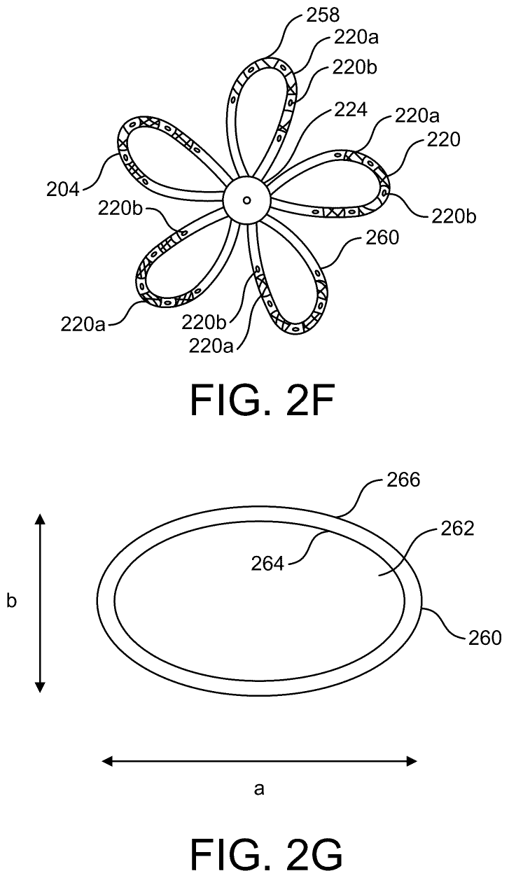

FIG. 2A is a side view of an embodiment of a mapping and ablation catheter 200, which corresponds with catheter 105 of FIG. 1. The catheter 200 can assume a plurality of configurations including a collapsed configuration and an expanded configuration. FIG. 2A illustrates the catheter 200 in the collapsed configuration. The catheter 200 includes an electrode array 202 comprising a set of splines 204 at a distal end 206 of the catheter 200 and a handle 208 at a proximal end 210 of the catheter 200. Each spline of the set of splines 204 includes one or more electrodes 220 formed on a surface of the spline. The electrodes can be configured as ablation electrodes for performing an ablation, sensing electrodes for performing mapping or detection of physiological signals, either ablation or sensing electrodes, or a combination of ablation electrodes and sensing electrodes. In the illustrated examples, the set of splines 204 include ablation electrodes 220a (that are illustrated as ring electrodes around the spline) and sensing electrodes 220b (that are illustrated as patch electrodes). Each spline of the set of splines 204 can be configured to include a flexible curvature so as to rotate, or twist and bend and form a petal or tear drop-shaped curve. The catheter 200 includes a catheter shaft 212 defining a longitudinal axis. The shaft 212 includes a distal region 214 and a proximal region 216. A shaft lumen extends longitudinally through the shaft 212 from the distal region 214 to the proximal region 216 along the longitudinal axis. In some embodiments, a diameter of the catheter shaft 212 is between about 6 French and about 15 French. In certain embodiments, the catheter shaft 212 has a diameter of about 12 French. In some embodiments, the catheter shaft 212 has a length of between about 85 cm and about 135 cm. In certain embodiments, the catheter shaft has a diameter of about 115 cm.

In some examples, the distal cap 224 can include a guidewire lumen, and a guidewire can extend along the longitudinal axis in the shaft lumen and through the guidewire lumen in the distal cap 224. The distal cap 224 includes an atraumatic shape in the illustration and can include an electrical component in some embodiments. The set of splines 204 extend from the distal region 214 of the shaft 212. In exemplary embodiments, the set of splines 204 includes a proximal end 221 affixed to the distal region 214 of the shaft 212. The splines include distal ends 222 tethered or fixed to a distal cap 224 such that the set of splines 204 and distal cap 224 can translate relative to the catheter shaft 212 so as to expand and contract together. In various embodiments, the distal cap 224 translates longitudinally along the guidewire extending through the lumen of the shaft. The set of splines 204 are configured for translation along the longitudinal axis to transition between the collapsed configuration and the expanded configuration. In some examples, the set of splines are configurable in an intermediate configuration for use such as ablation and mapping. The intermediate configuration is between the collapsed configuration and the expanded configuration, and the set of splines can be arranged in, for example, a basket or spherical configuration.

The proximal region 216 of the catheter shaft 212 in some embodiments is coupled to a handle 208 at the proximal end 210 of the catheter 200. The handle 208 in includes a guidewire lumen 230 through the handle 208. The handle 208 is operably coupled to the set of splines 204 and the distal cap 224. For example, the handle 208 includes a translation member 232 disposed in the handle 208. The translation member 232 can include a knob 234, such as a thumb-activated slider, configured to be manipulated translationally or rotationally by a clinician. In the illustrated embodiment, the translation member 232 is configured for translation along the longitudinal axis by moving or sliding the knob 234 between a plurality of positions 236 to transition the electrode array 202 between a set of configurations including a collapsed configuration at a collapsed configuration position 236a, the intermediate or basket configuration at an intermediate configuration position 236b, and the expanded configuration at an expanded configuration position 236c. In another embodiment, the translation member 232 can be configured to rotated about the longitudinal axis to transition the catheter 200 between a lock state and an unlock state. The lock state can fix a translational position of the set of splines 204 and the distal cap 224 relative to the catheter shaft 212 and the unlock state can permit translation of the distal cap 224 and set of splines 204 relative to the catheter shaft 212. In some embodiments, the handle 208 includes a flush port (not shown) for saline irrigation. For example, a saline flow may be used to maintain a predetermined level of flow to prevent thrombus formation. The handle 208 can include a cable 238 having connector 240. In some embodiments, the cable 238 is relatively short (e.g., up to about one meter) to increase maneuverability and flexibility of the catheter 200. The connector 240 are configured to couple to an extension cable to connect the catheter 200 to a console, such as console 130, or other components. In some embodiments, connector 240 is electrically connected to electrical components such as the electrodes 220, impedance tracking sensors, a magnetic sensor, temperature sensor, a gyroscopic sensor, an accelerometer via leads disposed in the handle 208, shaft 212, or on the electrode array 202. In some embodiments, the cable 238 and connector 240 can be configured for optical or other signal types.

FIGS. 2B, 2C, and 2D illustrate the electrode array 202 at the distal end 206 of the catheter 200 corresponding with FIG. 2A. More particularly, FIG. 2B illustrates the electrode array 202 in a collapsed configuration, FIG. 2C illustrates the electrode array 202 in the intermediate, or basket, configuration, and FIG. 2D illustrates the electrode array 202 in the expanded configuration. In some embodiments, the catheter 200 includes a radiopaque portion (not shown) that may be fluoroscopically imaged to aid a clinician in positioning the catheter 200 within the patient. The illustrated radiopaque portion includes a radiopaque marker band disposed over the set of splines 204 at a portion extending beyond a distal region 214 of the catheter shaft 212. Additionally, or alternatively, one or more of a distal portion of the catheter shaft 212 and the distal cap 224 can include a radiopaque portion or the radiopaque portion may be formed on a surface of a spline. Additionally, or alternatively, the catheter can include an EAM electrode to aid the clinician in positioning the catheter 200. In the illustrated embodiments, a translatable shaft 262 extends from catheter shaft 212, such as from within a lumen of the catheter shaft 212 and is movable with respect to the catheter shaft 212. In embodiments, the translatable shaft 262 is included with the translation member 232 and mechanically coupled to the knob 234. As the knob 234 is moved from the collapsed configuration position 236a to the intermediate configuration position 235b to the expanded configuration position 236c, the translatable shaft 262 is pulled within the lumen of the catheter shaft 212, or retracted into the catheter shaft 212. As the knob 234 is moved from the expanded configuration position 236c to the intermediate configuration position 235b to the collapsed configuration position 236a, the translatable shaft 262 is pushed out of the lumen of the catheter shaft 212, or extended out of the catheter shaft 212. In the illustrated example, the translatable shaft 262 is coupled to the distal cap 224, and the translatable shaft 262 and distal cap 224 are configured to move together with respect to the catheter shaft 212. A distal end 222 of each spline 260 of the set of splines 204 is coupled to a distal end of the translatable shaft 262, such as via cap 224, and a proximal end 221 of each spline 260 of the set of splines 204 is coupled to a distal end of the catheter shaft 212. As the translatable shaft 262 is moved relative to the catheter shaft 212, such as via knob 234 on handle 208, each spline 260 of the set of splines changes shape. In the illustrated example, the translatable shaft 262 includes a lumen in communication with a lumen in the distal cap 224 such as a guidewire lumen or a sheath lumen to receive a guidewire 270 or a sheath for a guidewire, and the translatable shaft 262 can move with respect to the guidewire 270.

FIG. 2B illustrates the set of splines 204 of the electrode array 202 in the collapsed configuration in which the set of splines 204 define a longitudinally extending cylinder. For example, each of the splines 260 in the set of splines 204 in the collapsed configuration includes a concave curve 244 facing the longitudinal axis. The curve 244 may be such that the set of splines 204 can be advanced through a patient's vasculature. In an embodiment, the knob 234 of the translation member 232 is slid forward to the collapsed configuration position 236a and the translatable shaft 262 of the translation member 232 is correspondingly extended, such as fully extended, from the distal region 214 of the catheter shaft 212 to form the collapsed configuration.

FIG. 2C illustrates the set of splines 204 of the electrode array 202 in the intermediate configuration in which the set of splines 204 define a basket. The concave curve 244 facing the longitudinal axis is more pronounced than in the collapsed configuration of FIG. 2B. The curve 244 may be such that the set of splines 204 are operational to perform a mapping or ablation. In an embodiment, the knob 234 of the translation member 232 is slid to the intermediate configuration position 236b and the translatable shaft 262 of the translation member 232 is correspondingly partially-retracted into and partially-extended from the distal region 214 of the catheter shaft 212 to form the intermediate configuration.

FIG. 2D illustrates the set of splines 204 of the electrode array 202 in the expanded configuration in which the set of splines 204 define a flower. The curves of the splines 260 are most pronounced in the expanded configuration. The set of splines 204 are operational to perform a mapping or ablation. In an embodiment, the knob 234 of the translation member 232 is slid to the expanded configuration position 236c and the translatable shaft 262 of the translation member 232 is correspondingly retracted, such as fully retracted, into the distal region 214 of the catheter shaft 212 to form the expanded configuration. In the illustrated embodiment, the distal cap 224 is abutted against the catheter shaft 212 to form the expanded configuration.

FIG. 2E illustrates another view of the set of splines 204 in the expanded configuration, where each spline in the set of splines 204 includes a loop 250 having a first concave curve 252 facing the spline distal end 222 or distal cap 224, a second concave curve 254 facing the longitudinal axis, and a third concave curve 256 facing the distal region 214 of the shaft 212. Each loop 250 of the set of splines 204 may be described as a flower petal where the set of splines 204 in the expanded configuration forms a flower catheter. In the collapsed configuration, the distal cap 224 is spaced from the distal region 214 of the catheter shaft 212 at a first distance d₁. In the expanded configuration, the distal cap 224 is spaced from distal region 214 of the catheter shaft 212 at a second distance d₂, which is less than the first distance d₁. For example, the second distance can be less than about 8 mm. In some embodiments, a ratio of the first distance d₁ to the second distance d₂ may be between about 5:1 and about 25:1. In some embodiments, the set of splines 204 and the distal cap 224 is configured for translation along the longitudinal axis by up to about 60 mm.

Each of the splines of the set of splines 204 includes a spline apex 258, which is a region of the spline that includes a maximum radial dimension from the longitudinal axis when in the expanded configuration as indicated in FIG. 2E The spline apex 258 is generally related to region of the spline having a relatively low radius of curvature or bend radius and greater bending or curvature in the expanded configuration, which is generally repeatable, and thus the spline apex 258 relates to the same region or area of the spline regardless of the whether spline is in the collapsed configuration or an intermediate configuration. The spline apex 258 is also indicated on the spine in the collapsed configuration in FIG. 2B, even though the maximum radial dimension from the longitudinal axis in the collapsed configuration (or the maximum radial dimension from the longitudinal axis in an intermediate configuration) may correspond with a different region of the spline.

FIG. 2F is a front view of an embodiment of the catheter 200 in the expanded configuration. Each spline of the set of splines 204 coupled to a distal cap 224 and form a plurality of petal-like curves that together resemble a flower. In this manner, the set of splines 204 twist, bend, and bias away from the longitudinal axis when translated from the collapsed configuration and allow the set of splines 204 to conform to the geometry of an endocardial space more easily, and particularly adjacent to the opening of a pulmonary vein or antrum. When viewed from the front as in FIG. 2F, each spline of the set of splines 204 displays an angle between the proximal and distal ends of the curve of more than 180 degrees. In some embodiments, one or more splines of the set of splines 204 in the expanded configuration bias away from the longitudinal axis of the catheter shaft 212 by up to about 30 mm. In some embodiments, the set of splines 204 in the expanded configuration collectively define a cross-sectional diameter of between about 10 mm and about 50 mm. In some embodiments, the set of splines 204 in the expanded configuration collectively define a cross-sectional diameter between about 25 mm and about 35 mm. In an exemplary embodiment, the set of splines 204 in the expanded configuration collectively define a cross-sectional diameter of about 31 mm or about 35 mm.

In some embodiments, the set of splines 204 in can rotate about the longitudinal axis in a helical manner between the collapsed and expanded configurations and reverse-helical manner between the expanded and collapsed configurations. For instance, the set of splines rotates about the longitudinal axis as the set of splines, or distal cap 224 translates along the longitudinal axis. The helical rotation of the set of splines 204 biases the set of splines 204 towards transitioning to an expanded configuration forming a set of loops (e.g., petals) spaced-apart from each other. This may help prevent the set of splines from undesirably bunching together. In some embodiments, each spline of the set of splines 204 may have a helix angle of less than about 5 degrees relative to the longitudinal axis of the catheter shaft 212. The helix angle is the angle of a spline of the set of splines 204 relative to the longitudinal axis of the catheter shaft 212. In other embodiments, each spline of the set of splines 204 can have a helix angle of less than about 2 degrees relative to the longitudinal axis of the catheter shaft 212, such as a helix angle of less than about 1 degree relative to the longitudinal axis of the catheter shaft 212.

FIG. 2G illustrates a cross-sectional view of a spline 260 of the set of splines 204. Generally, a cross-section of each spline of the set of splines 204 in the embodiment has a shape of an ellipse. In some embodiments, the ellipse shape has a major axis length (a) between about 1 mm and about 4 mm and a minor axis length (b) between about 0.4 mm and about 3 mm. For example, the major axis length (a) of the ellipse is between about 1 mm and about 2.5 mm and the minor axis length (b) is between about 0.4 mm and about 1.2 mm. The minor axis intersects the longitudinal axis of the catheter shaft. These dimensions help the splines 204 resist kinking and bunching of the spines, and aid bending of the spline 260 into the expanded configuration (e.g., petal shape). For example, the shorter minor axis aids bending (e.g., buckling) of the spline in a radial direction and the longer major axis provides lateral rigidity to the spline 260. In some embodiments, each spline 260 of the set of splines 204 has a cross-sectional area between about 0.2 mm² and about 15 mm². In some embodiments, when the set of splines 204 transitions between the collapsed configuration and the expanded configuration, each spline 260 changes shape (e.g., compress, expand). For example, a length of the major axis (a) can increase in the transition from the collapsed configuration to the expanded configuration. In some embodiments, a spline 260 in the collapsed configuration has a first major axis length and in the second configuration has a second major axis length. A ratio of the first major axis length to the second major axis length can be between about 4:5 and about 1:4 in some examples.

In some embodiments, the set of splines may include between about three splines and about twenty splines. For example, the set of splines may include five splines as illustrated or eight splines. In one example, the set of splines 204 are constructed from a polyether block amide and, in some examples, is available under the trade designations PEBAX from Arkema, S.A., and VESTAMID E from Evonik Industries, AG. In some embodiments, each spline of the set of splines 204 defines a spline lumen, such as spline lumen 262 extending along a length of spline 260. The spline 260 includes an inner wall 264 defining the lumen 262 and an outer wall or outer surface 266. The spline lumen 262 can be closed at the distal ends 222 of the spline or by the distal cap 224. Electrical leads or electrical components can be disposed within the spline lumen 262, such as in a manner that permits bending, buckling, extending, or translating the splines in the set of splines 204. Electrodes 220 are exposed on the spline 260. In some embodiments, the set of splines 204 in the expanded configuration are arranged as a set of electrically isolated loops.

Electrodes 220 are disposed on the set of splines 204, such as each spline in the set of splines includes a set of outwardly-facing electrodes. Electrodes 220 include ablation electrodes and sensing electrodes, and each spline of the set of splines 204 can include a set of ablation electrodes 220a and a set of sensing electrodes 220b. In some embodiments, each spline includes a set of electrodes having from two electrodes to eight electrodes, such as four ablation electrodes and four sensing electrodes, or more. In embodiments, each electrode is spaced-apart from other electrodes on that spline and on other splines in the expanded configuration. The electrodes 220 include an atraumatic shape to reduce trauma to tissue. For example, the electrodes 220 have an atraumatic shape including a rounded, flat, curved, or blunted portion configured to contact endocardial tissue. In some embodiments, the electrodes 220 may be located along any portion of the spline distal to the catheter shaft 212. In some embodiments, an additional electrode, such as an additional electrode used for ablation, can be located on the shaft 212 in the distal region 214, such as to operate the catheter in a bipolar mode. For instance, the addition electrode, which can include a plurality of additional electrodes on the shaft, can be operated as one of the cathode or anode when ablation electrodes on the set of splines 204 are operated as the other of the anode or cathode. The electrodes 220 can have the same or different sizes, shapes, and/or location along respective splines. In some embodiments, each ablation electrode 220a may have a surface area between about 0.5 mm² and about 20 mm² and each sensing electrode 220b may have a surface area in that range or less than that range. In one embodiment, a sensing electrode 220b has a surface area of about 0.32 mm².

In some embodiments, ablation electrodes 220a are electrically coupled to insulated electrical leads (not sown), such as each spline in the set of splines is associated with an electrical lead, such as an electrically insulated electrical lead, electrically coupled to the ablation electrode on that spline. For example, each ablation electrode on that spline can be associated with one electrical lead, which is electrically coupled to each ablation electrode. In some embodiments, one electrical lead is coupled to one or more ablation electrodes, and each ablation electrode can be associated with one connected lead in some embodiments. In some examples, the electrical lead is disposed within the spline lumen 262. In some embodiments, the set of ablation electrodes for each spline are jointly wired. In some embodiments, the set of ablation electrodes for each spline are wired in series. For example, a set of four electrodes on a spline are electrically coupled together using a single lead. The electrical lead may be disposed within a spline lumen to electrically couple to an associated ablation electrode through a corresponding aperture in the spline. In one embodiment, each insulated electrical lead may be configured for sustaining a voltage potential of at least about 700 Volts without dielectric breakdown of the corresponding insulation.

In some embodiments, the ablation electrodes 220a are independently addressable, and the ablation electrodes 220a can be energized in any sequence using any pulse waveform sufficient to ablate tissue by irreversible electroporation. For example, different sets of ablation electrodes 220a can deliver different sets of pulses (e.g., hierarchical pulse waveforms). The size, shape, and spacing of the ablation electrodes on each spline and between the splines are configured to deliver contiguous/transmural energy to electrically isolate one or more pulmonary veins in some embodiments. In some embodiments, a first set of ablation electrodes of a first spline of the set of splines 204 is configured as an anode and a second set of ablation electrodes of a second spline of the set of splines is configured as a cathode. The first spline may be non-adjacent to the second spline. This can increase the spacing between the splines and help prevent a short-circuit. In some of these embodiments, the first set of ablation electrodes includes one ablation electrode and the second set of ablation electrodes includes at least two ablation electrodes. In some embodiments, alternate ablation electrodes are at the same electric potential, and likewise for all the other alternating ablation electrodes. Thus, ablation may be delivered rapidly with all ablation electrodes activated at the same time. For example, a spline having a set of anode ablation electrodes can be activated together to deliver pulse waveforms for irreversible electroporation. Ablation electrodes on other splines can be activated together as cathode ablation electrodes on their respective splines so as to form an anode-cathode pairing for delivery of pulse waveforms for irreversible electroporation. The anode-cathode pairing and pulse waveform delivery can be repeated sequentially over a set of such pairings. For example, the splines can be activated sequentially in a clockwise or counter-clockwise manner. As another example, the cathode splines can be activated sequentially along with respective sequential anode spline activation until ablation is completed. In embodiments where ablation electrodes on a given spline are wired separately, the order of activation within the electrode of each spline can be varied as well. For example, the ablation electrodes in a spline can be activated all at once or in a predetermined sequence.

In one example, the catheter 200 is configured for use in the endocardial space. A guidewire is inserted into a patient's vasculature and advanced into the heart. For instance, a distal portion of the guidewire is advance into a pulmonary vein proximate the ostium. A proximal end of the guidewire is received into a guidewire lumen in the distal cap 224 on the catheter 200, and the catheter 200 is advanced over the guidewire so as to be disposed over the guidewire during use. Within the heart, the electrode array 202 is translated from the collapsed configuration to the intermediate or the expanded configuration for use in ablation or mapping. For instance, the set of splines 204 are configured in the expanded configuration and disposed against the ostium for ablation. The set of splines in the expanded configuration, in one embodiment, are arranged as a set of non-overlapping loops.

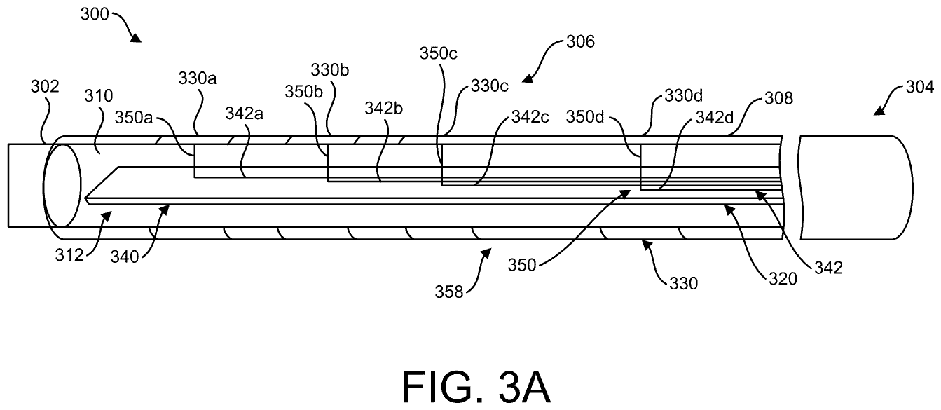

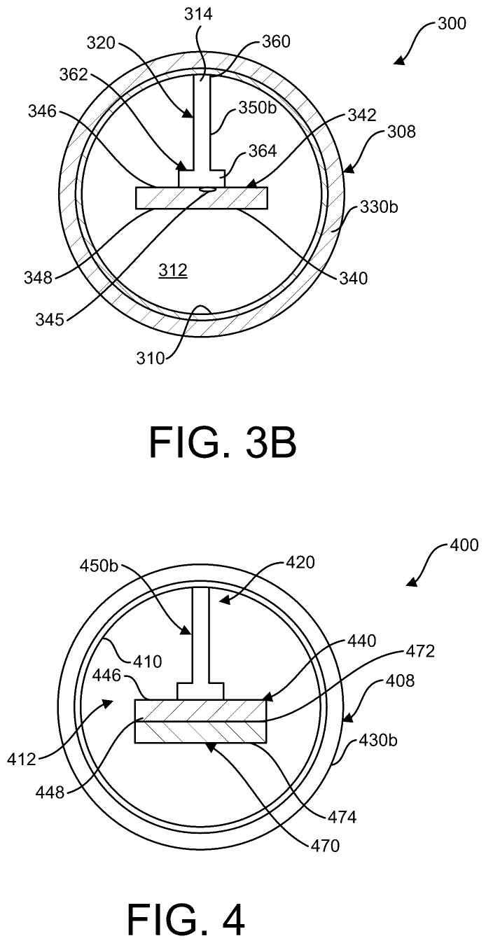

FIG. 3A illustrates cutaway view of a portion of a spline 300, which can correspond with spline 260 of the set of splines 204 on catheter 200. Spline 300 includes a distal region 302, which is configured to be coupled to the distal cap 224, a proximal region 304, which is configured to be coupled to the distal region 214 of the shaft 212, and an intermediate portion 306 between the distal and proximal regions 302, 304, which is configured to include electrodes 220 of catheter 200. The distal to proximal regions 302, 304 defines a longitudinal dimension of the spline 300 along an axis of the spline A. The cutaway view of the spline 300 illustrates an outer surface 308 and an inner wall 310, and the inner wall 310 defines a longitudinally extending major spline lumen 312.

As illustrated, the spline 300 includes an electrode assembly 320 formed on the spline 300. The electrode assembly 320 includes a plurality of longitudinally spaced-apart and outwardly-facing electrodes 330 disposed on and along the outer surface 308 of the spline 300 in the intermediate portion 306 of the spline. For example, the electrode assembly 320 includes a plurality of “n” spaced-apart electrodes 330 disposed on the surface 308 of the spline 300 (such as electrodes 1, . . . n). In the illustration, the spline 300 includes four spaced-apart electrodes 330 (n=4) configured as ring electrodes radially encircling the outer surface 308 of the spline 300. In other embodiments, the electrodes 330 can be configured as pad electrodes or other configurations. In some embodiments, the electrode assembly 320 can include ring electrodes and pad electrodes or any combination of two or more configurations. In the illustration, electrodes 330 include a distal-most electrode 330a in the intermediate portion of the spline 306, electrode 330b, electrode 330c, and a proximal-most electrode 330d in the intermediate portion of the spline 306.

The design of the electrode array 202 on catheter 200 provides for improved or optimized delivery of pulsed field ablation to the electrode assembly 320. Typically, insulated wires are used to couple to electrodes because of the relative ease of manufacturing the electrode array. This design, however, makes difficult the ability to include more than two individually addressable electrode groups on each spline due given the space requirements for electrical connections such as the insulated wires within the spline lumen. For example, the spline lumen includes space for two longitudinally extending wires introduced into the proximal end of the spline lumen. In one instance including four spaced-apart electrodes on the spline, a set of three electrodes of the four electrodes are electrically coupled to one of the two wires introduced into the spline lumen (such as the three electrodes are electrically coupled together in series) and the fourth electrode is electrically coupled to the other of the two wires introduced into the spline lumen. The spline lumen in a catheter of typical design dimensions does not accommodate three or more individually addressable electrode sets of one ore more electrodes.

The electrode assembly 320 includes a longitudinally extending flexible circuit 340 disposed within the major spline lumen 312. The flexible circuit 340 includes a plurality of longitudinally extending and electrically conductive lead traces 342 configured to carry an electrical signal, such as an electroporation ablation signal from console 130, a physiological signal received from tissue, or an electrical signal received from or provided to an electrical component in the electrode array 202 of catheter 200. The lead traces 342 are electrically isolated from one another. In one embodiment, the flexible circuit 340 comprises a layered structure that is typical of flexible circuits for use in medical device electrode assemblies. For instance, the flexible circuit 340 includes a dielectric base layer, an optional inner flexible adhesive layer over the base layer, a conductive trace layer including the conductive traces 340 over the adhesive layer (when present), and a dielectric upper layer over the conductive trace layer. The materials can be any conventional materials suitable for use in flexible circuits for medical devices, e.g., polyamides for the dielectric materials and copper for the conductive materials.

The flexible circuit 340 in one embodiment is formed to extend from at least the distal-most electrode (e.g., electrode 330a) to the handle 208 of catheter 200 where the flexible circuit 340 is electrically coupled to electrical connectors 240 in electrical cable 238. In another embodiment, the flexible circuit 340 is formed to extend from at least the distal-most electrode (e.g., electrode 330a) to the shaft 212 where it is electrically coupled to shaft electrical leads that are electrically coupled to the electrical connectors 240. The conductive lead traces 342 extend generally longitudinally along the spline 300 from at least the proximal region 304 to the corresponding electrode of the electrodes 330. For example, conductive lead trace 342a of sensing lead traces 342 is electrically coupled to electrode 330a and extends generally longitudinally along the spline 300 from at least the proximal region 304 to electrode 330a. Conductive lead trace 342b is electrically coupled to electrode 330b and extends generally longitudinally along the spline 300 from at least the proximal region 304 to electrode 330b. Conductive lead trace 342c is electrically coupled to electrode 330c and extends generally longitudinally along the spline 300 from at least the proximal region 304 to electrode 330c. Conductive lead trace 342d is electrically coupled to electrode 330d and extends generally longitudinally along the spline 300 from at least the proximal region 304 to sensing electrode 330d. In some examples, however, a conductive lead trace is coupled to more than one electrode of the plurality of electrodes 330, such that the two or more electrodes are electrically coupled together.

The electrode assembly 320 further includes a plurality of jumpers 350 electrically coupling the flexible circuit 340 to the plurality of electrodes 330. In the example, one of the plurality of jumpers 350 corresponds with one of the plurality of electrodes 330 and one of the plurality of lead traces 342. For instance, jumper 350a provides an electrical connection between conductive lead trace 342a and electrode 330a, jumper 350b provides an electrical connection between conductive lead trace 342b and electrode 330b, jumper 350c provides an electrical connection between conductive lead trace 342c and electrode 330c, and jumper 350d provides an electrical connection between conductive lead trace 342d and electrode 330d. In one example, jumpers 350 are used to space-apart the flexible circuit from the inner wall 310 of the spline 300. In one example, the jumpers 350 extend through an aperture in the spline 300 to mechanically connect with the electrodes 330 disposed on the outer surface 308 of the spline 300, such as an aperture underneath the ring electrode and hidden from view in the illustration. In some embodiments, the jumpers 350 are coupled to their corresponding conductive lead trace 342 via solder and coupled to their corresponding electrode 330 via a weld. In one example, the conductive lead trace of the conductive lead traces 342 extends through a dielectric layer of the flexible circuit 340 radially underneath its corresponding electrode of the electrodes 330 and forms an electrically conductive pad on an outer surface of the flexible circuit in which the conductive pad is suitable for attaching to the corresponding jumper 350. In some embodiments, the jumpers 350 are formed to be flexible, to allow bending or buckling of the spline 300 and electrode assembly 320, such as jumpers 350 comprising springs or electrical wires. In other embodiments, the jumpers 350 are formed to be generally rigid, such as to rigidly space-apart the flexible circuit 340 from the inner wall 310 of the spline, such as jumpers comprising rigid wires or standoffs, in which the standoffs are integrally formed with the electrodes 330 or included as part of the flexible circuit 340. In other examples, the jumpers can be semi-rigid or semi-flexible to incorporate features and advantages of rigid and flexible jumpers.

FIG. 3B illustrates a cross sectioned view of the spline 300 coupled to the electrode assembly 320 consistent with the configuration of the intermediate portion 306 of the spline 300 without the cutaway of FIG. 3A. Spline 300 includes outer surface 308 and the inner wall 310 defining the major spline lumen 312. The electrode assembly 320 includes a ring electrode 330b, as an example, disposed around the outer surface 308 of the spline 300, flexible circuit 340 disposed within the major spline lumen 312, and jumper 350b electrically coupled to the electrode 330b and the corresponding conductive lead trace (such as conductive lead trace 342b in FIG. 3A) of the flexible circuit 340. In the illustrated example, spline 300 includes an aperture 314 extending from the outer surface 308 to the inner wall 310 to allow jumper 350b to extend from the electrode 330b into the major spline lumen 312. The jumper 350b includes a radially distal end 360 that is welded to a radially inner surface of the electrode 330b. The electrode 330b covers the aperture 314 in its entirety, and the electrode 330b is swaged onto the spline 300 to seal the aperture 314. The flexible circuit 340 includes a first major surface 346 and an opposite second major surface 348. In the illustrated example, the first and second major surfaces 346, 348 are generally planar and the first major surface 346 faces the aperture 314. In one embodiment, the flexible circuit 340 is of a layered configuration, and the first and second major surfaces 346, 348 comprise a dielectric material with the conductive traces 342 disposed between the dielectric materials. In this embodiment, the conductive trace 342b corresponding with the electrode 330b is electrically coupled through the first major surface to an electrically conductive pad 345 disposed on the first major surface 346 of the flexible circuit 340. The electrically conductive pad 345 is soldered to a radially proximal end 362 of the jumper 342b. The illustrated example includes an electrically conductive shoulder 364 formed at the radially proximal end 362 of the jumper 350b to facilitate electrical and mechanical connection of the jumper 350b to the pad 345. In another example, the spline 300 can be overmolded onto the flexible circuit 340 and electrode 330b after the electrodes 330 have been coupled to the flexible circuit 340 so as not to form a well-defined major spline lumen 312.

FIG. 4 illustrates a cross sectioned view of an embodiment of a spline 400 having a shape memory strut 470 disposed within the spline 400. The spline 400 is coupled to an electrode assembly 420 consistent with the configuration of the intermediate portion 306 of the spline 300 and electrode assembly 320 of FIG. 3B. In embodiments, each spline of the set of splines 204 includes a shape memory strut 470 disposed within the associated spline. Spline 400 includes outer surface 408 and the inner wall 410 defining the major spline lumen 412. The electrode assembly 420 includes a ring electrode 430b, as an example, disposed around the outer surface 408 of the spline 400, flexible circuit 440 disposed within the major spline lumen 412, and jumper 450b electrically coupled to the electrode 430b and the corresponding conductive lead trace of the flexible circuit 440. The flexible circuit 440 includes a first major surface 446 and an opposite second major surface 448. In the illustrated example, the first and second major surfaces 446, 448 are generally planar and the first major surface 446 faces the jumper 450b. In one embodiment, the flexible circuit 440 is of a layered configuration, and the first and second major surfaces 446, 448 comprise a dielectric material with the conductive traces disposed between the dielectric materials. In this embodiment, the conductive trace corresponding with the electrode 430b is electrically coupled to the jumper 450b. The shape memory strut 470, which extends longitudinally along the spline such as from a distal region to a proximal region, includes a first major strut surface 472 and a second major strut surface 474. The first and second major strut surfaces 472 and 474 are planar in the illustrated example. The second major surface 448 of the flexible circuit 440 is affixed to the first major surface 472 of the strut 470, such as via an adhesive, so that the flexible circuit 440 moves with the strut 470. (In other embodiments, the cross sections of a strut in the major spline lumen 412 are curvilinear. In one example, the cross section of the strut is circular or elliptical, in which case the strut includes a single, continuous major surface having first and second sides, the first side facing the flexible circuit 440 such that the second side 448 of the flexible circuit 440 is affixed to the first side of the strut having the circular or elliptical cross section.) The shape memory strut 470 extends through the spline 400 such as from a distal region of the spline to the proximal region of the spline. In one example, the strut 470 can include a distal end coupled to the distal cap 324 and a proximal end coupled to the shaft 212.

The shape memory strut 470 forms, among other things, as a primary structural support of the spline 400, and, in embodiments in which a strut 470 is included in each spline of the set of splines 204, primarily defines the mechanical characteristics of the electrode array 202. In embodiments, the strut 470 is formed from a superelastic material (metal or polymer) to provide desired mechanical/structural properties to the electrode array 202. In embodiments, the strut 470 is formed from a superelastic metal alloy, e.g., a nickel-titanium alloy. Forming the strut 470 from a superelastic material such as a nickel-titanium alloy facilitates configuring the strut 470 to assume its desired unconstrained shape due to the shape memory properties of the material, while providing sufficient flexibility necessary to collapse or expand the electrode array 202. The shape memory strut 470 predictably governs the shape of the electrode array 202 through translation between the collapsed configuration, intermediate configuration, and expanded configuration. Further, in some examples, the shape memory strut 470 can serve to reduce deployment force during translation and improve planarity. In one embodiment, the shape memory strut is shape set in the intermediate configuration such as to form a basket. In another embodiment, the shape memory strut is shape set in the expanded configuration such as to form the petals of the flower. In still another embodiment, the shape memory strut 470 is shape set in the collapsed configuration. In embodiments, each shape memory strut 470 of a plurality of shape memory struts in the electrode array 202 can be selectively configured along their lengths to tune the mechanical characteristics of the electrode array 202.

In one example, the spline 400 does not include a major spline lumen. Rather, the spline 400 can be formed via overmolding or other manufacturing techniques onto the flex circuit 440 and strut 470. In such an example, the flexible circuit 440 is affixed to the strut 470, and the spline is formed over the flexible circuit and strut 470 assembly.

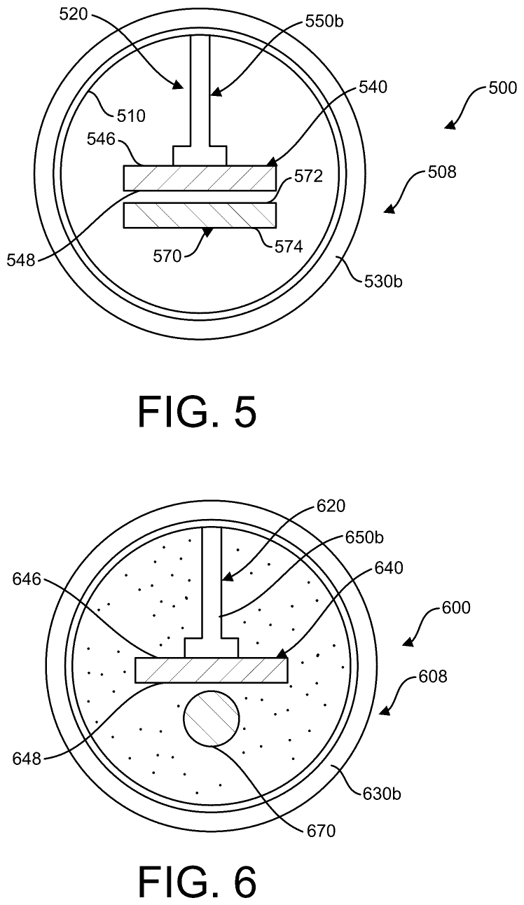

FIG. 5 illustrates a cross sectioned view of an embodiment of a spline 500 having a shape memory strut 570 disposed within the spline 500. The spline 500 is coupled to an electrode assembly 520 consistent with the configuration of the intermediate portion 306 of the spline 300 and electrode assembly 320 of FIG. 3B. In embodiments, each spline of the set of splines 204 includes a shape memory strut 570 disposed within the associated spline, which, similar to shape memory strut 470 of FIG. 4, forms the primary structural support of the spline 500. Spline 500 includes outer surface 508 and the inner wall 510 defining the major spline lumen 512. The electrode assembly 520 includes a ring electrode 530b, as an example, disposed around the outer surface 508 of the spline 500, flexible circuit 540 disposed within the major spline lumen 512, and jumper 550b electrically coupled to the electrode 530b and the corresponding conductive lead trace of the flexible circuit 540. The flexible circuit 540 includes a first major surface 546 and an opposite second major surface 548. In the illustrated example, the first and second major surfaces 546, 548 are generally planar and the first major surface 546 faces the jumper 550b. In one embodiment, the flexible circuit 540 is of a layered configuration, and the first and second major surfaces 546, 548 comprise a dielectric material with the conductive traces disposed between the dielectric materials. In this embodiment, the conductive trace corresponding with the electrode 530b is electrically coupled to the jumper 550b. The shape memory strut 570, which extends longitudinally along the spline such as from a distal region to a proximal region. The shape memory strut 570 forms, among other things, as a primary structural support of the spline 500, and, in embodiments in which a strut 570 is included in each spline of the set of splines 204, primarily defines the mechanical characteristics of the electrode array 202. In embodiments, the strut 570 is formed from a superelastic material (metal or polymer) to provide desired mechanical/structural properties to the electrode array 202. The strut 570 includes first and second major strut surfaces 572 and 574, which are planar in the illustrated example. In another example, the cross section of the strut 570 is curvilinear, such as the strut 570 is configured as a rod extending along the longitudinal axis of the spline 500. Characteristics of the strut 570, such as shape set, are described above with respect to strut 470.

The flexible circuit 540 is offset from and not directly affixed to the strut 570, such as via an adhesive, so that the flexible circuit 540 moves generally independently from the strut 570. In this example, deployment force used to translate the electrode array 202 between the collapsed configuration and expanded configurations can be reduced and planarity can be improved. With the flexible circuit 540 offset from and not directly laminated to the strut 570, the strut 570 governs the shape of the spline 500 and can make the spline 500 more pliable or softer. Further, the selection of the planar sheet type or rod type strut 570 can influence shape set.

FIG. 6 illustrates a cross sectioned view of an embodiment of a spline 600 having a rod type shape memory strut 670 disposed within the spline 600. The spline 600 is coupled to an electrode assembly 620 consistent with the configuration of the intermediate portion 306 of the spline 300 and electrode assembly 320 of FIG. 3B. In embodiments, each spline of the set of splines 204 includes a shape memory strut 670 disposed within the associated spline, which, similar to shape memory strut 470 of FIG. 4, forms the primary structural support of the spline 600. Spline 600 includes outer surface 608. In the example, the spline 600 does not include a major spline lumen, such as the major spline lumen in previously described examples. Rather, the spline 600 can be formed via overmolding or other manufacturing techniques onto the flex circuit 640 and strut 670.

The electrode assembly 620 includes a ring electrode 630b, as an example, disposed around the outer surface 608 of the spline 600, flexible circuit 640 disposed within the spline 600, and jumper 650b electrically coupled to the electrode 630b and the corresponding conductive lead trace of the flexible circuit 640. The flexible circuit 640 includes a first major surface 646 and an opposite second major surface 648. In the illustrated example, the first and second major surfaces 646, 648 are generally planar and the first major surface 646 faces the jumper 650b. In one embodiment, the flexible circuit 640 is of a layered configuration, and the first and second major surfaces 646, 648 comprise a dielectric material with the conductive traces disposed between the dielectric materials. In this embodiment, the conductive trace corresponding with the electrode 630b is electrically coupled to the jumper 650b.

The shape memory strut 670 extends longitudinally along the spline such as from a distal region to a proximal region. The shape memory strut 670 forms, among other things, as a primary structural support of the spline 600, and, in embodiments in which a strut 670 is included in each spline of the set of splines 204, primarily defines the mechanical characteristics of the electrode array 202. In embodiments, the strut 670 is formed from a superelastic material (metal or polymer) to provide desired mechanical/structural properties to the electrode array 202. The strut 670 is configured as a rod extending along the longitudinal axis of the spline 600 but can also include other configurations such as a sheet type strut having planar major strut surfaces. Characteristics of the strut 670, such as shape set, are described above with respect to strut 470. The flexible circuit 640 is offset from and not directly affixed to the strut 670, such as via an adhesive, but both the flexible circuit 640 and strut 670 are disposed in the overmolded material of the spline 600. With the flexible circuit 640 offset from and not directly laminated to the strut 670, the strut 670 governs the shape of the spline 600 and can make the spline 600 more pliable or softer. The amount of offset between the flexible circuit 640 and strut 670 can influence shape and stiffness of the spline 600.

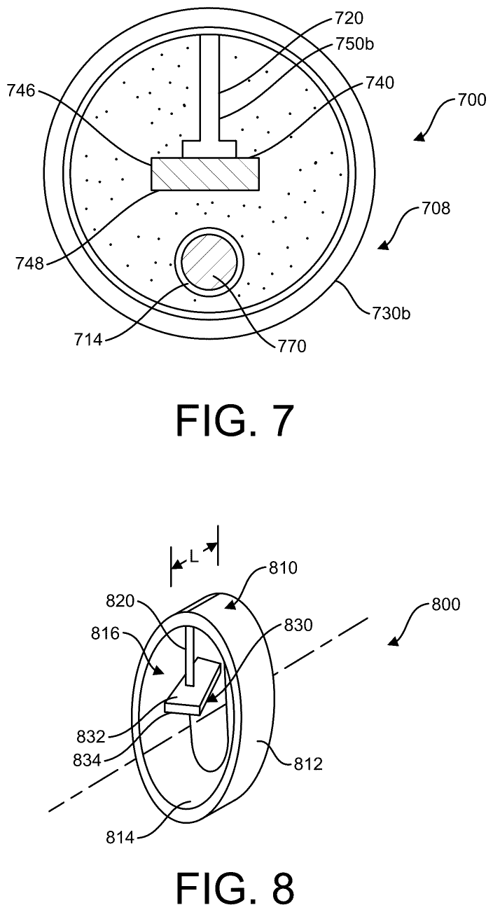

FIG. 7 illustrates a cross sectioned view of an embodiment of a spline 700 having a rod type shape memory strut 770 disposed within the spline 700. The spline 700 is coupled to an electrode assembly 720 consistent with the configuration of the intermediate portion 306 of the spline 300 and electrode assembly 320 of FIG. 3B. In embodiments, each spline of the set of splines 204 includes a shape memory strut 770 disposed within the associated spline, which, similar to shape memory strut 470 of FIG. 4, forms the primary structural support of the spline 700. Spline 700 includes outer surface 708. In the example, the spline 700 does not include a major spline lumen, such as in some of the previously described examples. Rather, the spline 700 can be formed via overmolding or other manufacturing techniques onto the flex circuit 740. The spline 700, however, includes an inner wall 710 forming strut lumen 714. The rode type shape memory strut 770 is disposed within the strut lumen 714 and not affixed to the inner wall 710.

The electrode assembly 720 includes a ring electrode 730b, as an example, disposed around the outer surface 708 of the spline 700, flexible circuit 740 disposed within the spline 700, and jumper 750b electrically coupled to the electrode 730b and the corresponding conductive lead trace of the flexible circuit 740. The flexible circuit 740 includes a first major surface 746 and an opposite second major surface 748. In the illustrated example, the first and second major surfaces 746, 748 are generally planar and the first major surface 746 faces the jumper 750b. In one embodiment, the flexible circuit 740 is of a layered configuration, and the first and second major surfaces 746, 748 comprise a dielectric material with the conductive traces disposed between the dielectric materials. In this embodiment, the conductive trace corresponding with the electrode 630b is electrically coupled to the jumper 750b.

The shape memory strut 770 extends longitudinally along the spline 700 within the strut lumen 714 such as from a distal region to a proximal region. The shape memory strut 770 forms, among other things, as a primary structural support of the spline 700, and, in embodiments in which a strut 770 is included in each spline of the set of splines 204, primarily defines the mechanical characteristics of the electrode array 202. In embodiments, the strut 770 is formed from a superelastic material (metal or polymer) to provide desired mechanical/structural properties to the electrode array 202. The strut 770 is configured as a rod extending along the longitudinal axis of the spline 700 but can also include other configurations such as a sheet type strut having planar major strut surfaces. Characteristics of the strut 770, such as shape set, are described above with respect to strut 470. The flexible circuit 740 is offset from and not directly affixed to the strut 770 and the strut 770 is not affixed to the material of the spline 700. With the strut 770 spaced apart from the spline 700, the strut 770 still governs the shape of the spline 700 and can make the spline 600 more pliable or softer, but the shape memory strut 770 can move with respect to the spline 700 to reduce fatigue on the strut 770 and allow the strut 770 to achieve its shape set.

FIG. 8 illustrates an example electrode 800 for use with a spline in the set of splines in catheter 200. In one example, the electrode 800 can be configured to be used with splines 300, 400, 500, 600, and 700. Electrode 800 can be configured as dedicated ablation electrode, dedicated sensing electrode, or electrodes configured for ablation and sensing, and is formed from a suitable electrically conductive material. The electrode 800 in the example is configured as a ring electrode having an atraumatic ring 810 having a longitudinal ring length L, a ring outer surface 812, and a ring inner surface 814 defining a ring lumen 816. The electrode 800 is configured to be disposed around an outer surface of the spline with the ring inner surface 814 of the electrode 800 against the spline. The electrode 800 further includes an electrically conductive standoff 820 integrally formed into the ring inner surface 814 extending radially into the ring lumen 816. The standoff 820 in the example is configured as a radially-extending post. An electrically conductive shoulder 830 is electrically coupled to the standoff 820. The shoulder 830 includes a first major shoulder surface 832 facing radially outwardly and electrically and mechanically coupled to the standoff 820 and a second major shoulder surface 834, facing opposite the first major shoulder surface 832, configured to electrically couple to a electrical interconnect extending longitudinally within the spline, such as flexible circuit described above. The standoff 820 and shoulder 830 form a “T” shape in the example when viewed from an end although other configurations are possible.

The shoulder 830 can have a longitudinal shoulder length, measured in a direction of the longitudinal ring length L, and a lateral shoulder length, measured in a lateral direction of the spline, such as perpendicular to the longitudinal length. In one example, the longitudinal shoulder length is longer than longitudinal ring length, such as the shoulder 820 extends longitudinally out from underneath the ring 810 on each side of the electrode 800. In the illustrated example, the shoulder 830 is configured to extend longitudinally out from underneath one side of the ring 810 but not the other. In this example, the shoulder 830 can easily be electrically coupled to an electrical interconnect, such as the flexible circuit, prior to the spline being overmolded over the shoulder 830 and interconnect. In one example, the electrode 800 is extruded in a unitary piece. In another example, the standoff 820 is welded to the ring 810 and the shoulder 830. Other configurations are contemplated.

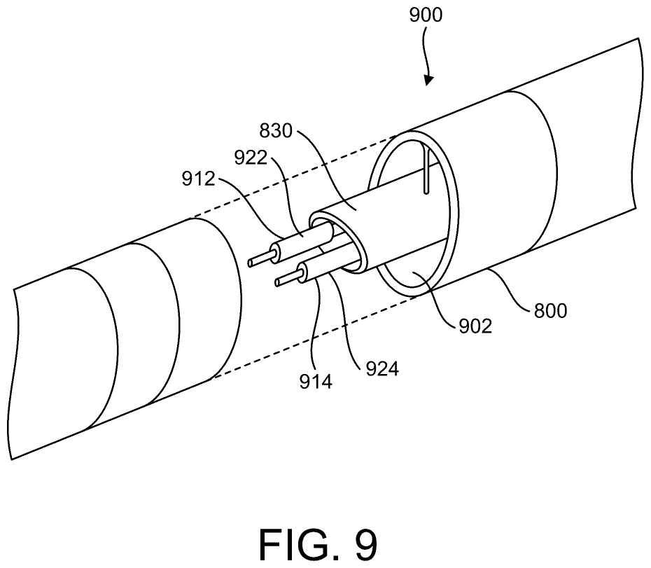

FIG. 9 illustrates a sectioned view of a spline 900 having an electrode constructed in accordance with electrode 800. The spline 900 in the illustrated example includes a spline lumen 902 although other configurations are possible. In the example, the shoulder 830 is electrically coupled to an electrical interconnect comprising a plurality of electrically insulated wires, such as wires 912, 914. For example, the shoulder 830 is electrically coupled to one of wires 912, 914 and another electrode on the longitudinal length of the spline is electrically coupled to the other of wires 912, 914. In one instance, one of wires 912, 914, such as wire 912, has insulation 922 stripped in the longitudinal region of the shoulder 830 to expose an electrical conductor underneath the insulation 922. The exposed electrical conductor is electrically coupled to the shoulder 830. In the illustrated example, the shoulder 830 is crimped to the exposed conductor of wire 912. In other examples, the shoulder 830 can be welded or soldered to the exposed conductor of wire 912. Wire 914 is not electrically coupled to the shoulder 830. Rather, the shoulder 830 is merely crimped to the insulation 924 of wire 914 to hold the wire in place, and no electrical connection is made. In this example, the wires 912, 914 are maintained in the center or near the center of the spline 900, which can yield an improved mechanical performance of the spline, such as during translation and in the expanded configuration of the electrode assembly, and reduce strain on the wires 912, 914. In another electrode in the lumen, wire 914 is electrically and mechanically coupled to the shoulder, and the other wire, wire 912 is not electrically coupled but mechanically coupled to the shoulder, such as in a similar manner. This configuration provides for two individually addressable electrode groups on each spline 900 if the space allows for two insulated wires within the spline 900. In one instance including four spaced-apart electrodes on the spline, a set of three electrodes of the four electrodes are electrically coupled to one of the two wires, such as wire 912, such as three electrodes electrically coupled together in parallel, and the fourth electrode is electrically coupled to the other of the two wires, such as wire 914, introduced into the spline lumen 902.

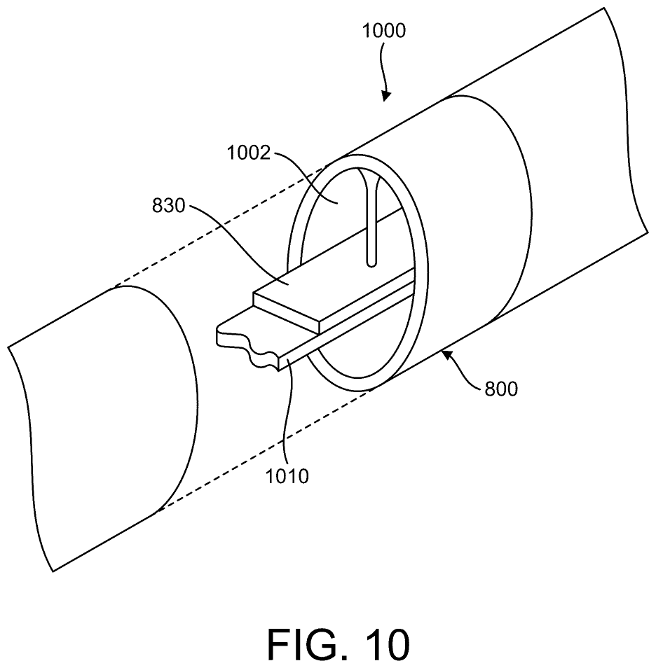

FIG. 10 illustrates a sectioned view of a spline 1000 having an electrode constructed in accordance with electrode 800. The spline 1000 in the illustrated example includes a spline lumen 1002 although other configurations are possible. In the example, the shoulder 830 is electrically coupled to an electrical interconnect 1010 comprising a longitudinally extending, electrically conductive, shape memory material, such as a nickel-titanium alloy. The shoulder 830 is electrically coupled, such as by welding or soldering, directly to the nickel-titanium alloy electrical interconnect 1010. The electrical interconnect can be in various forms including a plate and rod, with electrically conductive surfaces exposed along the entire length or generally covered in an electrical insulative material having selectively exposed surfaces to connect to the shoulder of each electrode of a plurality of electrodes. The electrical interconnect 1010 can be configured to include features such as flags to readily connect to the shoulders 830. The electrical interconnect 1010 can be welded or soldered directly to the shoulder. In one example, the interconnect 1010 is able to carry the same signal to each electrode in the spline 1000. Via the conductive shape memory material, each spline in the set of splines can be shape set in a selected configuration, such as in the intermediate configuration of the basket or the expanded configuration of the flower.

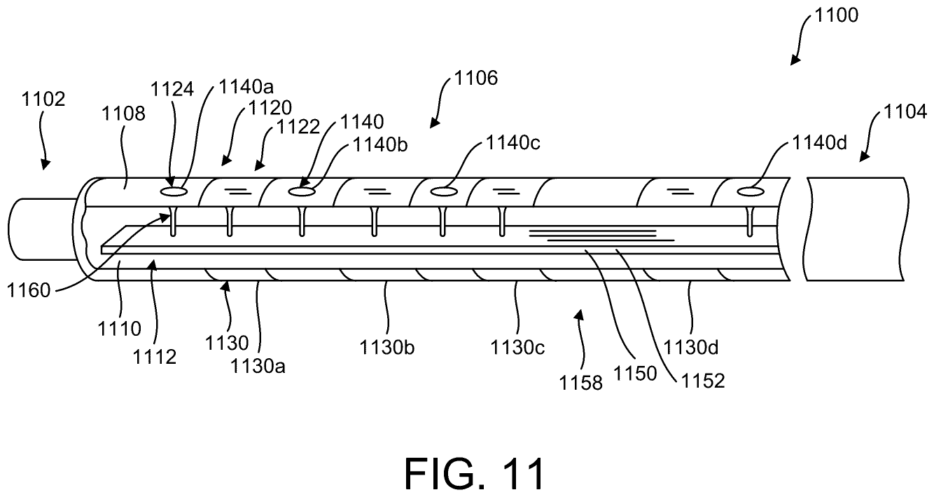

FIG. 11 illustrates a portion of a spline 1100, which can correspond with spline 260 of the set of splines 204 on catheter 200. Spline 1100 includes a distal region 1102, which is configured to be coupled to the distal cap 224, a proximal region 1104, which is configured to be coupled to the distal region 214 of the shaft 212, and an intermediate portion 1106 between the distal and proximal regions 1102, 1104, which is configured to include electrodes 220 of catheter 200. The distal to proximal regions 1102, 1104 defines a longitudinal dimension of the spline 1100 along an axis of the spline. In some embodiments, the spline 1100 includes a spline outer surface 1108 and a spline inner surface 1110 and defines a longitudinally extending major spline lumen 1112. FIG. 11 further illustrates a region of the spline 1100 corresponding with a spline apex 1158, which, when included in the catheter 200, will provide the region of the spline 1100 that includes the maximum radial dimension from the longitudinal axis in the expanded configuration.

As illustrated, the spline 1100 includes a plurality of electrode assemblies 1120 formed on the spline 1100. The plurality of electrode assemblies 1120 includes an ablation electrode assembly 1122 and a sensing electrode assembly 1124. The ablation electrode assembly 1122 includes a plurality of longitudinally spaced-apart and outwardly-facing ablation electrodes 1130 disposed on and along a surface 1108 of the spline 1100. For example, the ablation electrode assembly 1122 includes a plurality of “n” spaced-apart ablation electrodes 1130 disposed on the surface 1108 of the spline 1100 (such as ablation electrodes 1, . . . n). In the illustration, the spline 1100 includes four spaced-apart ablation electrodes 1130 (n=4) configured as ring electrodes radially encircling the surface 1108 of the spline 1100. In other embodiments, the ablation electrodes 1130 can be configured as pad electrodes or other configurations. In some embodiments, the ablation electrode assembly 1120 can include ring electrodes and pad electrodes or any combination of two or more configurations. In the illustration, ablation electrodes 1130 include a distal-most electrode 1130a in the intermediate portion of the spline 1106, electrode 1130b, electrode 1130c, and a proximal-most electrode 1130d in the intermediate portion of the spline 1106.

The sensing electrode assembly 1124 includes a plurality of longitudinally space-apart and outwardly-facing sensing electrodes 1140 disposed on and along a surface 1108 of the spline 1100. For example, the sensing electrode assembly 1124 includes a plurality of “m” spaced-apart sensing electrodes 1140 disposed on the surface 1108 of the spline 1100 (such as sensing electrodes 1, . . . m). In the illustration, the spline 1100 includes four spaced-apart sensing electrodes 1130 (m=4) configured as pad electrodes on the surface 1108 of the spline 1100. The pad electrodes can be formed as ellipses having a major axis and a minor axis. In the illustrated example, the major axis extends with the longitudinal dimension of the spline 1100. The sensing electrodes 1140 are also spaced-apart and electrically isolated from the ablation electrodes 1130 on the spline 1100. In the illustrated example, a distal-most sensing electrode 1140a in the intermediate portion 1106 of the spline 1100 is distal to a distal-most ablation electrode 1130a in the intermediate portion 1106 (in the collapsed configuration or longitudinally along the spline 1100). Further, a proximal-most sensing electrode 1140d in the intermediate portion 1106 is proximal to a proximal-most ablation electrode 1130d in the intermediate portion 1106. Additional sensing electrodes 1140 are interspersed between the spaced-apart ablation electrodes 1130 along the longitudinal dimension such as one sensing electrode of the sensing electrodes 1140 is disposed between two ablation electrodes of the ablation electrodes 1130 in the longitudinal dimension. For example, sensing electrode 1140b is disposed between ablation electrode 1130a and ablation electrode 1130b, and sensing electrode 1130c is disposed between ablation electrode 1130b and ablation electrode 1130c. Furthermore, the spline apex 1158 in the illustrated embodiment is disposed between two spaced-apart ablation electrodes adjacent to the spline apex 358, such as ablation electrodes 1130c, 1130d, and no sensing electrodes of sensing electrodes 1140 are disposed on the spline 1100 between ablation electrodes adjacent to the spline apex 1158, such as ablation electrodes 1130c, 1130d.

The electrode assembly 1120 includes a longitudinally extending flexible circuit 1150 disposed within the major spline lumen 1112. The flexible circuit 1150 includes a plurality of longitudinally extending and electrically conductive lead traces 1152 configured to carry an electrical signal, such as an electroporation ablation signal from console 130, a physiological signal received from tissue, or an electrical signal received from or provided to an electrical component in the electrode array 202 of catheter 200. The lead traces 1152 are electrically isolated from one another. In one embodiment, the flexible circuit 1150 comprises a layered structure that is typical of flexible circuits for use in medical device electrode assemblies, such as described above. The flexible circuit 1150 in one embodiment is formed to extend from at least the distal-most electrode (e.g., sensing electrode 1140a) to the handle 240 of catheter 200 where the flexible circuit 1150 is electrically coupled to electrical connectors 240 in electrical cable 238. In another embodiment, the flexible circuit 1150 is formed to extend from at least the distal-most electrode to the shaft 212 where it is electrically coupled to shaft electrical leads that are electrically coupled to the electrical connectors 240. The conductive lead traces 1152 extend generally longitudinally along the spline 1100 from at least the proximal region 1104 to the corresponding electrode of the ablation electrodes 1130 and sensing electrodes 1140.

In one embodiment, each of the plurality of electrodes 1130, 1140 corresponds with one of the plurality of electrically conductive lead traces 1152 on the flexible circuit 1140. In some examples, however, a conductive lead trace is coupled to more than one electrode, such that the two or more electrodes are electrically coupled together via a lead trace of the plurality of lead traces 1152. In one embodiment, the flexible circuit comprises more than one flexible circuit, such as an ablation flexible circuit having a plurality of ablation lead traces configured for electrically coupling to the ablation electrodes 1130 and a sensing flexible circuit having a plurality of sensing lead traces configured for electrically coupling to the sensing electrodes 1140. Other configurations are contemplated, such as a first flexible circuit having first set of ablation lead traces and first set of sensing lead traces and second flexible circuit having a second set of ablation lead traces a second set of sensing lead traces. In one example, the first and second flexible circuits can be arranged such that the corresponding lead traces face opposite radial directions within the major spline lumen 1112. For instance, the multiple flex circuits can be coupled to opposite major surfaces of a shape memory strut within the major spline lumen 1112. In another embodiment, a single flexible circuit can include all the lead traces on one major surface of flexible circuit, which is folded in half with a bend, fold, or crease extending longitudinally along the major spline lumen 1112 such that a first group of lead traces face in one outwardly radial direction and a second group of lead traces face in an opposite outwardly radial direction. In one embodiment, the flex circuit can be bent around a shape memory strut within the major spline lumen 1112.

The electrode assembly 1120 further includes a plurality of jumpers 1160 electrically coupling the flexible circuit 1150 to the plurality of ablation electrodes 1130 and the plurality of sensing electrodes 1140.

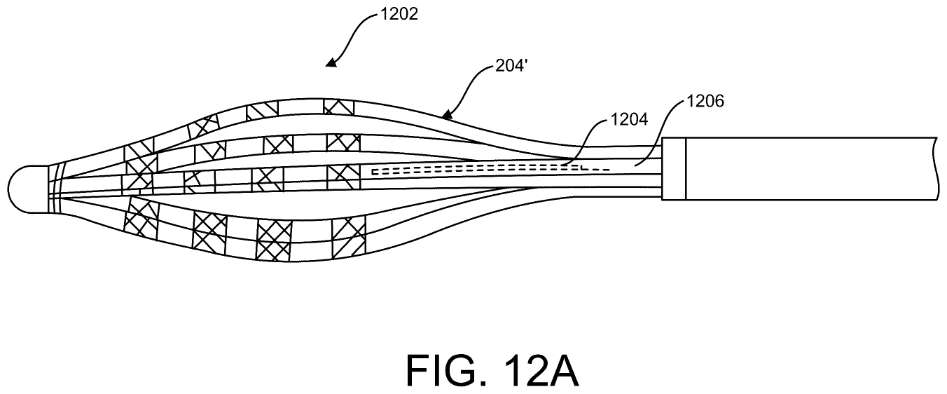

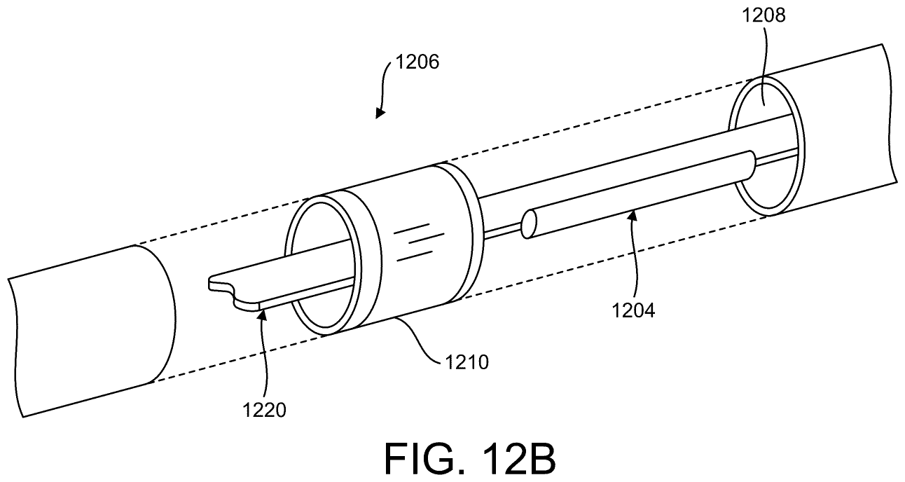

FIG. 12A illustrates an embodiment of an electrode array 1202, configured for use at the distal end of the catheter 200, in which the electrode array 1202 is in the collapsed configuration. The electrode array 1202 can correspond with electrode array 202, wherein electrode array 1202 includes a navigation sensor 1204 (shown in phantom) disposed within a spline 1206 of the plurality of splines 204′. The navigation sensor 1204 in the embodiment is disposed proximal to the proximal most electrode on an intermediate portion of the spline 1206 in an area having a relatively large radius of curvature in the expanded configuration such that the spline is able to translate from the collapsed configuration into the petal in the expanded configuration. In one example, the navigation sensor 1204 is a five degree of freedom navigational sensor able to provide electrical signals indicative orientation and position and thus of the shape and position of the electrode assembly 1202.

FIG. 12B illustrates a cutaway view of the spline 1206 having major spline lumen 1208 and a plurality of electrodes 1210 disposed on a surface 1212 of the spline 1206. The spline lumen 1208 includes a flexible circuit 1220 having electrically conductive lead traces in electrical communication with the handle 208 of the catheter as described above. The flexible circuit 1220, via conductive lead traces, is electrically coupled to electrodes 1210 and to the navigational sensor 1204. In one embodiment, the navigational sensor 1204 is directly coupled to the flexible circuit 1220, such as an electrical connector on the navigational sensor 1204 is welded to an electrically conductive pad on the surface of the flexible circuit 1220 in electrical communication with a lead trace.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A catheter for ablating cardiac tissue through irreversible electroporation, the catheter comprising:
an elongated shaft having a distal region, the elongated shaft defining a longitudinal axis; and
a set of splines extending from the distal region of the shaft, the set of splines configured for translation along the longitudinal axis to transition between a collapsed configuration and an expanded configuration, wherein each spline forms a loop in the expanded configuration;
wherein each spline of the set of splines includes an electrode assembly comprising a flexible circuit within the spline, the flexible circuit including a plurality of lead traces electrically coupled to a plurality of electrodes on a surface of the spline.

2. The catheter of claim 1, wherein the electrode assembly includes a plurality of electrode assemblies formed on each spline, the plurality of electrode assemblies including an ablation electrode assembly and a sensing electrode assembly, wherein the flexible circuit includes a set of ablation lead traces of the plurality of lead traces electrically coupled to a plurality of ablation electrodes of the plurality of electrodes and wherein the flexible circuit includes a set of sensing lead traces of the plurality of lead traces electrically coupled to a plurality of sensing electrodes of the plurality of electrodes.

3. The catheter of claim 2, wherein a distal-most sensing electrode is distal to a distal-most ablation electrode and a proximal-most sensing electrode is on the spline.

4. The catheter of claim 2, wherein each spline includes an equal number of sensing electrodes and ablation electrodes.

5. The catheter of claim 1, wherein each of the plurality of electrodes is individually addressable.

6. The catheter of claim 1, wherein the electrodes of the plurality of electrodes are configurable as at least one of an ablation electrode and a sensing electrode.

7. The catheter of claim 1, wherein the spline defines a major spline lumen, and the flexible circuit is disposed within the major spline lumen.

8. The catheter of claim 1, wherein that spline includes a shape memory strut.

9. The catheter of claim 8, wherein the shape memory strut is shape set in the expanded configuration.

10. The catheter of claim 1, wherein the set of splines in the expanded configuration are arranged as a set of electrically isolated loops.

11. The catheter of claim 1, wherein the set of splines are arranged to helically rotate about the longitudinal axis.

12. The catheter of claim 1, and further comprising a distal cap coupled to a distal portion of each spline of the set of splines.

13. The catheter of claim 12, wherein the loop each spline forms includes a first concave curve facing the distal cap, a second concave curve facing the longitudinal axis, and a third concave curve facing the distal end of the shaft.

14. The catheter of claim 1, wherein the electrodes are outwardly-facing on the surface of the spline.

15. A catheter for ablating cardiac tissue through irreversible electroporation, the catheter comprising:
an elongated shaft having a distal region, the elongated shaft defining a longitudinal axis;
a set of splines extending from the distal region of the shaft; and
a distal cap coupled to a distal portion of each spline of the set of splines, the set of splines configured for translation along the longitudinal axis to transition between a collapsed configuration and an expanded configuration,
wherein in the expanded configuration, each spline forms a loop having a first concave curve facing the distal cap, a second concave curve facing the longitudinal axis, and a third concave curve facing the distal end of the shaft;
wherein each spline of the set of splines includes an electrode assembly formed on that spline, the electrode assembly comprising a flexible circuit within that spline, the flexible circuit including a plurality of lead traces electrically coupled to a plurality of outwardly-facing electrodes on a surface of the spline.

16. The catheter of claim 15, wherein the electrode assembly includes a plurality of electrode assemblies formed on each spline, the plurality of electrode assemblies including an ablation electrode assembly and a sensing electrode assembly, wherein the flexible circuit includes a set of ablation lead traces of the plurality of lead traces electrically coupled to a plurality of ablation electrodes of the plurality of electrodes and wherein the flexible circuit includes a set of sensing lead traces of the plurality of lead traces electrically coupled to a plurality of sensing electrodes of the plurality of electrodes.

17. The catheter of claim 16, wherein a distal-most sensing electrode is distal to a distal-most ablation electrode and a proximal-most sensing electrode is on the spline and wherein each spline includes an equal number of sensing electrodes and ablation electrodes.

18. A catheter for ablating cardiac tissue through irreversible electroporation, the catheter comprising:
an elongated shaft having a distal region, the elongated shaft defining a longitudinal axis;
a set of splines extending from the distal region of the shaft; and
a distal cap coupled to a distal portion of each spline of the set of splines, the set of splines configured for translation along the longitudinal axis to transition between a collapsed configuration and an expanded configuration, wherein in the expanded configuration, each spline forms a loop having a first concave curve facing the distal cap, a second concave curve facing the longitudinal axis, and a third concave curve facing the distal end of the shaft;
wherein each spline of the set of splines includes an electrode assembly formed on the spline, the electrode assembly including an ablation electrode assembly and a sensing electrode assembly, the electrode assembly comprising a flexible circuit within the spline, the flexible circuit including a plurality of lead traces electrically coupled to a plurality of electrodes on a surface of the spline, wherein the flexible circuit includes a set of ablation lead traces of the plurality of lead traces electrically coupled to a plurality of ablation electrodes of the plurality of electrodes and wherein the flexible circuit includes a set of sensing lead traces of the plurality of lead traces electrically coupled to a plurality of sensing electrodes of the plurality of electrodes.

19. The catheter of claim 18, wherein the set of splines in the expanded configuration are arranged as a set of electrically isolated loops.

20. The catheter of claim 18, wherein the set of splines are arranged to helically rotate about the longitudinal axis.
